(19) United States
(12) Patent Application Publication
(10) Pub. No.: US 2002/0198189 A1
(43) Pub. Date: Dec. 26, 2002

(54) New use

(76) Inventors: Olivier Besencon, Allschwil (CH); Rolf Olsson, Gnesta (SE); Johan Ohman, Uppsala (SE); Patrizia Caldirola, Uppsala (SE)

Correspondence Address: JEFFREY D. HSI, Fish & Richardson P.c., 225 Franklin Street, Boston, MA, 02110-2804, US

(21) Appl. No.: 10/045,319

(22) Filed: Nov. 9, 2001

Related U.S. Application Data
(60) Provisional application No. 60/252,156, filed on Nov. 20, 2000.

(30) Foreign Application Priority Data
Nov. 9, 2000 (SE) 0004101-2

(51) Int. Cl. A61K 31/55; A61K 31/4745; C07D 471/02

(52) U.S. Cl. 514/215; 514/303; 540/578; 546/118

(57) ABSTRACT

A method of treatment or prophylaxis of SSAO-mediated complications in mammals including humans, comprising administering to a patient in need of such treatment a therapeutically effective amount of a compound of Formula (I):

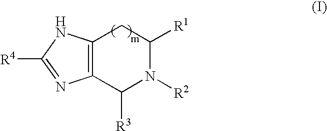

in which R¹, R², R³ and R⁴ are as described in the specification.

### CROSS REFERENCE TO RELATED APPLICATIONS

This application claims priority from Swedish Patent Application No. 0004101-2, filed Nov. 9, 2000, and U.S. Provisional Patent Application Serial No. 60/252,156, filed Nov. 20, 2000. These applications are incorporated herein by reference in their entirety.

### FIELD OF INVENTION

The present invention relates to use of 4-alkyl-5-alkoxycarbonyl-4, 5, 6, 7-tetrahydroimidazo[4,5-c] pyridine derivatives for the manufacture of medicaments for, or treatment or prophylaxis of semicarbazide-sensitive amine oxidase (SSAO)-mediated complications.

### BACKGROUND OF INVENTION

Semicarbazide-sensitive amine oxidase, SSAO, is a monoamine oxidase that recently has been suggested to be responsible for microvascular complications in diabetic patients. Nephropathy, neuropathy and retinopathy represent the end results of microvascular complications in both insulin dependent diabetes mellitus (IDDM) and non-insulin dependent diabetes mellitus (NIDDM). Heart attack, angina, strokes, amputations, blindness and renal failure are clinical events that represent the end point of the clinical study. Endothelial cells dysfunction may precede the diabetic state of complications. The SSAO enzyme is located in the vascular smooth muscles, retina, kidney and the cartilage tissues, and in the circulating blood (Yu, P. H. Deamination of methylamine and angiopathy; toxicity of formaldehyde, oxidative stress and relevance to protein glycoxidation in diabetes. J. Neural. Transm. Suppl., 1998, 52, 201) and has been found to be overactive in diabetic patients (Ekblom, J. Potential therapeutic value of drugs inhibiting semicarbazide-sensitive amine oxidase: vascular cytoprotection in diabetes mellitus. Pharmacol. Res., 1998, 37, 87). SSAO oxidizes a primary amine into the corresponding aldehyde with the help of the non-proteogenic amino acid topaquinone (Scheme 1). Oxidation of the aminophenol form of topaquinone into the quinone form, in order to close the catalytic cycle, is catalyzed by Cu(II), present in the active site of the enzyme. Hydrogen peroxide and ammonia are produced. The general catalytic cycle is very similar to the one observed for other monoamine oxidases (Scheme 1). The higher activity of SSAO as well as the higher concentration of its natural substrates, methylamine and aminoacetone, in diabetic patients, would lead to a higher production of formaldehyde, methylglyoxal and hydrogen peroxide. These products are known to be highly cytotoxic for the endothelial cell layer and might lead to the observed microvascular complication in diabetic patients (Yu, P. H. Deamination of methylamine and angiopathy; toxicity of formaldehyde, oxidative stress and relevance to protein glycoxidation in diabetes. J. Neural. Transm. Suppl., 1998, 52, 201). The inhibition of SSAO-mediated reactions is therefore a strategy that could be beneficial for a variety of pathological conditions.

Scheme 1: SSAO's catalytic cycle. The active site is represented schematically by the topaquinone residue as well as by a basic amino acid residue B⁻ essential for the activity.

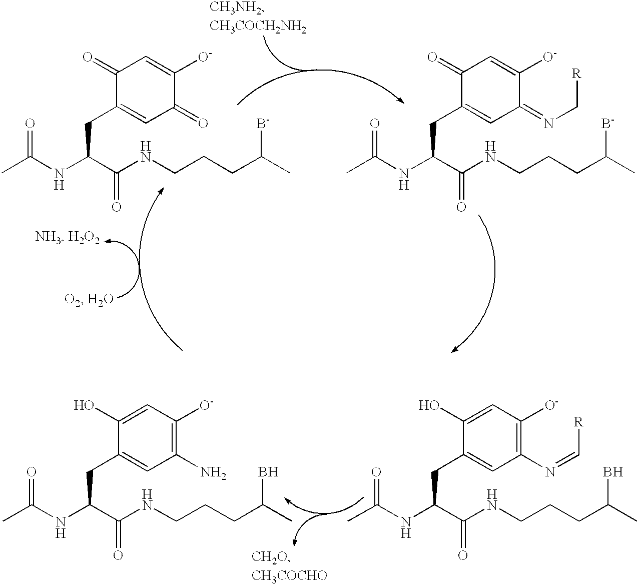

### SUMMARY OF THE INVENTION

According to the invention a method of treatment or prophylaxis of SSAO-mediated complications in mammals including humans is provided. In one aspect, the invention relates to a method of treatment or prophylaxis of SSAO-mediated complications in mammals including humans, comprising administering to a patient in need of such treatment a therapeutically effective amount of a compound of Formula (I):

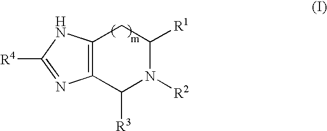

or a pharmaceutically acceptable salt thereof, wherein

R¹ is

(a) H, or

(b) CONH—R⁵;

R² is

(a) COOR⁵,

(b) COR⁵,

(c) CONH—R⁵,

(d) CSNH—R⁵, or

(e) H;

R³ is

(a) H,

(b) C₁₋₈ alkyl, or

(c) (CH₂)ₙAr;

R⁴ is

(a) H,

(b) Ar, or

(c) C₁₋₈ alkyl; and

R⁵ is

(a) H,

(b) (CH₂)ₙAr,

(c) (CH₂)ₙOAr,

(d) C₁₋₈ alkyl containing 0-2 oxygen atoms and optionally substituted with 0-5 halogen atoms, or

(e) a polyether chain having the formula (CH₂)ₓO(CH₂)_{y}O(CH₂)_{z}CH₃;

n is an integer 0 to 4;

m is an integer 0 to 2;

x and y are integers 2 to 4;

z is an integer 0 to 3;

Ar is phenyl, 1-naphthyl or 2-naphthyl, unsubstituted optionally mono-or poly-substituted with electrodonating groups, halogen, C₁₋₆ alkyl, CF₃, hydroxyl, C₁₋₆ alkoxyl, OCF₃, CN, NO₂, phenyloxyl, benzyloxyl, optionally substituted phenyl, alkylsulfonyl, C₁₋₆ alkenyl, —NH₂, R⁷NH—, R⁷, R⁷N—, C₁₋₆ alkylcarboxyl, formyl, C₁₋₆ alkyl-CO—NH—, aminocarbonyl (R R⁷—N—CO—), SR⁷ wherein R⁷ is simultaneously or alternatively H or C₁₋₆ alkyl; cynnamoyl, unsubstituted or optionally substituted benzyl; 1,1-diphenylethyl, a monocyclic or bicyclic heterocyclic ring (furyl, pyrrolyl, triazolyl, diazolyl, oxazolyl, thiazolyl, oxadiazolyl, isothiazolyl, isoxazolyl, thiadiazolyl, pyridyl, pyrimidyl, pyrazinyl, thienyl, imidazolyl, pyrazolyl, indolyl, quinolinyl, isoquinolinyl, benzofuryl, benzothienyl, benzoxadiazolyl which are unsubstituted or optionally mono or di-substituted with halogen, C₁₋₆ alkyl); 2, or 3, or 4-pyridyl or a 5 to 7-membered unsaturated or partially or completely saturated heterocyclic ring each containing 1 to 4 heteroatoms selected from oxygen, nitrogen or sulfur where nitrogen containing heterocycles may contain H or C₁₋₆ alkyl or CF₃—CO— at the nitrogen atoms where such a substitution is allowed.

In another aspect, the method comprises administering to a patient (e.g., mammal, human) in need of such treatment a therapeutically effective amount of a compound of Formula (I):

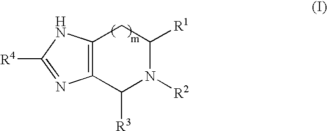

or a pharmaceutically acceptable salt thereof, wherein

R¹ is

(a) H, or

(b) CONH—R⁵;

R² is

(a) COOR⁵,

(b) COR⁵,

(c) CONH—R⁵, or

(d) CSNH—R⁵;

R³ is

(a) H,

(b) C₁₋₈ alkyl, or

(C) (CH₂)ₙAr;

R⁴ is

(a) H,

(b) Ar, or

(c) C₁₋₈ alkyl; and

R⁵ is

(a) H,

(b) (CH₂)ₙAr,

(c) (CH₂)ₙOAr,

(d) C₁₋₈ alkyl, or

(e) a polyether chain having the formula (CH₂)ₓO(CH₂)_{y}O(CH₂)_{z}CH₃;

n is an integer 0 to 4;

m is an integer 0 to 2;

x and y are integers 2 to 4;

z is an integer 0 to 3;

Ar is phenyl, 1-naphthyl or 2-naphthyl, unsubstituted optionally mono-or poly-substituted with halogen, C₁₋₆ alkyl, CF₃, hydroxyl, C₁₋₆ alkoxyl, OCF₃, CN, NO₂, phenyloxyl, benzyloxyl, optionally substituted phenyl, alkylsulfonyl, C₁₋₆ alkenyl, —NH₂, R⁷NH—, R⁷, R⁷N—, C₁₋₆ alkylcarboxyl, formyl, C₁₋₆ alkyl-CO—NH—, aminocarbonyl (R⁷, R⁷—N—CO—), SR⁷ wherein R⁷ is simultaneously or alternatively H or C₁₋₆ alkyl; cynnamoyl, unsubstituted or optionally substituted benzyl; 1,1-diphenylethyl, a monocyclic or bicyclic heterocyclic ring (furyl, pyrrolyl, triazolyl, diazolyl, oxazolyl, thiazolyl, oxadiazolyl, isothiazolyl, isoxazolyl, thiadiazolyl, pyridyl, pyrimidyl, pyrazinyl, thienyl, imidazolyl, pyrazolyl, indolyl, quinolinyl, isoquinolinyl, benzofuryl, benzothienyl, benzoxadiazolyl which are unsubstituted or optionally mono or di-substituted with halogen, C₁₋₆ alkyl); 2, or 3, or 4-pyridyl or a 5 to 7-membered unsaturated or partially or completely saturated heterocyclic ring each containing 1 to 4 heteroatoms selected from oxygen, nitrogen or sulfur where nitrogen containing heterocycles may contain H or C₁₋₆ alkyl or CF₃—CO— at the nitrogen atoms where such a substitution is allowed.

Another aspect of the invention relates to methods of inhibiting SSAO activity in a mammal, including humans, comprising administering to a patient (e.g., mammal, human) in need of such treatment a therapeutically effective amount of a compound of any of the formulae delineated herein, including those of Formula I and those delineated in the tables herein. In another aspect, the methods of inhibiting SSAO activity using compounds delineated herein are useful in instances (e.g., treatment or prevention of diseases, including those herein) where modulation (e.g., downregulation, or control of overproduction) of abnormal levels of SSAO or overactive levels of SSAO in the subject (e.g., patient, mammal, human) are present.

The term “C₁₋₈ alkyl” denotes a saturated or unsaturated, straight, branched or cyclic alkyl group having from 1 to 8 carbon atoms. Examples of said C₁₋₈ alkyl include methyl, ethyl, n-propyl, iso-propyl, n-butyl, iso-butyl, sec-butyl, t-butyl, and straight- and branched-chain pentyl, hexyl, septyl and octyl, optionally substituted with 0-5 halogen atoms.

The term “halogen” shall mean fluorine, chlorine, or bromine.

Electrodonating groups (e.g., electron-donating groups) are known in the art, including in J. March, Advanced Organic Chemistry, 2^{nd} Ed., McGraw-Hill Book Company, New York, (1977), and subsequent editions thereof.

### DETAILED DESCRIPTION OF THE INVENTION

According to the present invention it has been found that 4-alkyl-5-alkoxycarbonyl-4,5,6,7-tetrahydroimidazo[4,5-c] pyridine derivatives of Formula (I) are potent compounds for inhibiting SSAO-mediated reactions. 4-alkyl-5-alkoxycarbonyl-4,5,6,7-tetrahydroimidazo[4,5-c] pyridine derivatives are known from GB 2 158 440 and U.S. Pat. No. 4,223,146. In the GB application the compounds are disclosed to have anti-viral activity. The compounds in the U.S. patent are useful as antiulcer agents and as inhibitors of gastric secretion.

In Formula (I) preferred substituents are as follows:

R¹ is H, CO—NH2, R³ is C₁₋₃ alkyl or benzyl and R² is COOR⁵ and R⁵ is

1) H or a linear, branched or cyclic C₁₋₈ alkyl which can be saturated or not, containing 0-2 oxygen atoms and optionally substituted with 0-5 halogen atoms;

2) (CH₂)ₙAr, where n 0-3 and Ar is a phenyl group or a phenyl group substituted with electrodonating groups and/or halogen atoms.

Preferred compound of Formula (I) are:

benzyl 4-methyl-1,4,6,7-tetrahydro-5H-imidazo[4,5-c]pyridine-5-carboxylate;

benzyl 4-ethyl-1,4,6,7-tetrahydro-5H-imidazo[4,5-c]pyridine-5-carboxylate trifluoroacetate;

benzyl 4-propyl-1 ,4,6,7-tetrahydro-5H-imidazo[4,5-c]pyridine-5-carboxylate trifluoroacetate;

2,2,2-trichloroethyl 4-ethyl-1,4,6,7-tetrahydro-5H-imidazo[4,5-c]pyridine-5-carboxylate; and

benzyl (4S,6S)-6-(aminocarbonyl)-4-ethyl-1,4,6,7-tetrahydro -5H-imidazo[4,5-c]pyridine-5-carboxylate trifluoroacetate.

The compounds of the Formula (I) can form acid addition salts with acids such as conventional pharmaceutically acceptable acids, for example maleic, hydrochloric, hydrobromic, phosphoric, acetic, fumaric, salicylic, citric, lactic, mandelic, tartaric and methanesulfonic.

Compounds of Formula (I) may also form solvates such as hydrates and the invention also extends to these forms. When referred to herein, it is understood that the term “compound of Formula (I)” also includes these forms.

Certain compounds of Formula (I) are capable of existing in stereoisomeric forms including diastereomers and enantiomers and the invention extends to each of these stereoisomeric forms and to mixtures thereof including racemates. The different stereoisomeric forms may be separated one from the other by the usual methods. Any given isomer may be obtained by stereospecific or asymmetric synthesis. The invention also extends to any tautomeric forms and mixtures thereof.

Preferably the compounds of Formula (I) are used for treatment or prophylaxis of SSAO mediated vascular complications and for insulin dependent diabetes mellitus and non-insulin dependent diabetes mellitus.

The compounds used in the invention are prepared according to known methods. The compounds can be prepared as follows:

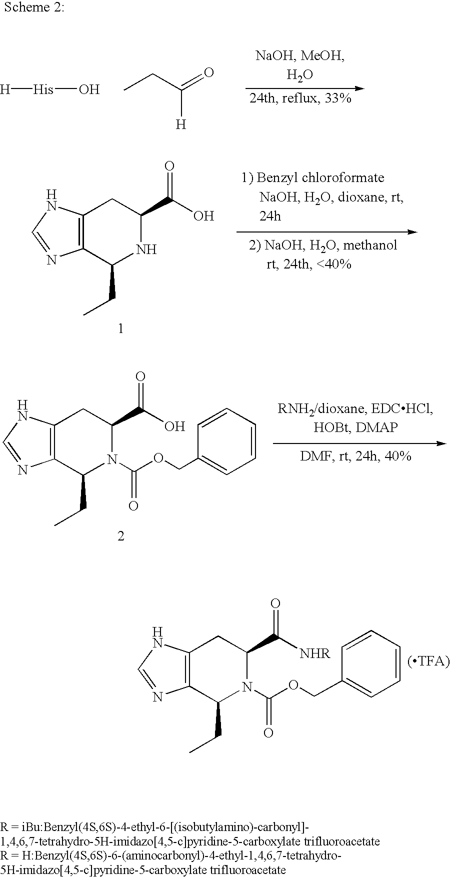

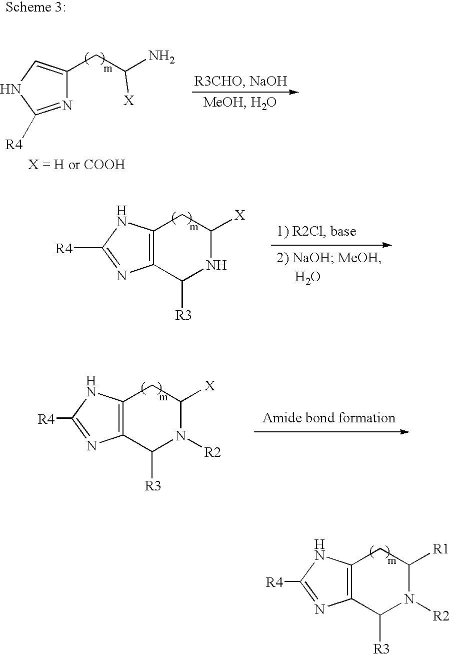

The starting materials are commercially available or can be prepared following known procedures.

According to the present invention the compounds for treatment of SSAO mediated complications can conveniently be administered in a pharmaceutical composition containing the compound in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15^{th} Ed., 1975). The compounds and compositions can be administered orally, parenterally (for example, by intravenous, intraperitoneal or intramuscular injection), topically, or rectally.

Useful dosages of the compounds of Formula (I) can be determined by comparing their in vitro activity, and in vivo activity in animal models. Methods for the extrapolation of effective dosages in mice, and other animals, to humans are known in the art; for example, see U.S. Pat. No. 4,938,949.

The compound can be administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may be presented in a single dose or as divided doses administered at appropriate intervals. The compositions can be administered orally, sublingually, transdermally, or parenterally at dose levels of about 0.01 to about 150 mg/kg, preferably about 0.1 to about 50 mg/kg, and more preferably about 0.1 to about 30 mg/kg of mammal body weight.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

The invention will now be illustrated with the following examples, which however, are for illustrative purposes are not intended to limit the scope of the invention.

The following abbreviations are used:

aq: aqueous; br: broad; DIPEA: diisopropylethylamine; DMAP: 4-(N,N-dimethylamino)pyridine; ECAO: Esterichia Coli amine oxidase; EDC.HCl: N-ethyl-N′-3(dimethylamino)propylcarbodiimide hydrochloride; EI: electron impact; eq: equivalent; EtOAc: ethyl acetate; HOBt: hydroxybenzotriazol; HTS: high throughput screening;; Mp: melting point; HRMS: high resolution mass spectrum; org: organic; RP—HPLC: reversed-phase high pressure liquid chromatography; SAR: structure activity relationship; sat: saturated; TLC: thin layer chromatography.

Experimental:

Solvents were purchased from Merck or Riedel-de Haen. Chemicals and reagents were purchased from Aldrich, Lancaster or Fluka. The buffer solution (pH 9) was bought from Merck (catalogue number 1.09461.1000, boric acid/KCl/NaOH). TLCs were run using Silicagel60 F₂₅₄ plates purchased from Merck. TLCs were analysed by UV or stained with a solution of KMnO₄ in water. Flash chromatography was run using Silicagel 60 (230-400 mesh) from Merck. Parallel flash chromatography was run on a Foxy-200 system from Isco Inc. with 108-disposable columns for FC from Isco Inc. RP—HPLCs were run on a Gilson system, using a 119 UV-detector (214 nm or 254 nm), a 805 manometric module, a 305- and a 306-pumps and a Vydac C₁₈-column (218TP1022); H₂O+0.1% TFA/CH₃CN were used as eluents. Mps were measured with a Gallenkamp apparatus and were uncorrected. NMR spectra were recorded on a Varian Inova 400 instrument. (EI)-MS spectra were recorded on a JMS SX-102A mass spectrometer (Jeol, Tokyo, Jpn) at 70 eV or on an Autospec-oaTOF Micromass Manchester instrument at 70 eV. HRMS spectra were recorded on a LCT Micromass instrument with flow injection-electrospray positive mode; quaternary ammonium salts were used as references. Reactions were followed by MS, using a Platform I Micromass instrument, Manchester, with an electrospray positive and negative mode flow injection. Elemental analysis was run on an Elementar Vario EL instrument.

General Procedure I (GPI):

A solution of histidine or histamine, NaOH and aldehyde was prepared in water and MeOH, and was heated under reflux for 24 h. After cooling the solution to room temperature, then to 0° C., aq. conc. HCl was added. See the specific examples for the work-up procedures.

General Procedure II (GPII):

A solution of tetrahydroimidazopyridine and K₂CO₃ in CHCl₃ and water was cooled to 0° C. The chloroformate, resp. the acyl chloride was added dropwise. The mixture was stirred for 24 h while warming up to room temperature. The phases were separated, the org. phase dried over Na₂SO₄ and the solvent removed under reduced pressure. The residue was dissolved or suspended in MeOH (3 mL/mmol of starting material) and aq. 1M NaOH (2 mL/mmol of starting material) was added. After 1 h, the mixture was worked-up and purified, see specific examples.

Carboxylic Acid Intermadiate (Scheme 2)

(4S,6S)-4-Ethyl-4,5,6,7-tetrahydro-3H-imidazo[4,5-c]pyridine-6-carboxylic Acid:

According to GPI with histidine (15.5 g, 0.100 mol), NaOH (24 g, 0.60 mol), water (100 mL), MeOH (400 mL) and propionaldehyde (20 mL, 0.276 mol). The solution was then acidified to pH 7-8 with aq. conc. HCl (60 mL) and the solvent was removed under reduced pressure. After drying the residue thoroughly under high vacuum, the oil was triturated with hot EtOH and filtered (3×). The filtrate was evaporated under reduced pressure and the residue crystallized from EtOH/water. (4S,6S)-4-Ethyl-4,5,6,7-tetrahydro-3H-imidazo[4,5-c]pyridine-6-carboxylic acid was isolated as a white powder (6.44 g, 33%). ¹H-NMR and NOE-measurement showed a cis/trans ratio of 9:1. Mp=242-4° C. ¹H—NMR (400 MHz, D₂O; only cis-stereoisomer described): δ=7.75 (s, 1H); 4.43 (m, br., 1H); 4.05 (dd, J₁=12.1 Hz, J₂=5.2 Hz, 1H); 3.81 (dd, J₁=16.5 Hz, J₂=5.3 Hz, 1H); 3.00 (ddd, J₁=16.5 Hz, J₂=12.1 Hz, J₃=2.4 Hz, 1H); 2.27 (m, 1H); 1.91 (m, 1H); 1.14 (t, J=7.6 Hz, 3H). ¹³C—NMR (100 MHz, D₂O): δ=173.28 (s); 137.27 (d); 129.83 (s); 124.10 (s); 57.98 (d); 56.20 (d); 25.17 (t); 23.79 (t); 9.51 (q). MS (EI): m/z=194 (M⁺—H₂, 2%); 166 (61%); 148 (9%); 120 (100%); 107 (10%); 93 (10%). HRMS: Calc. for C₉H₁₃N₃O₂: M⁺=195.1008; found: M⁺=195.0000.

Intermediate 2 (Scheme 2)

(4S,6S)-5-[(Benzyloxy)carbonyl]-4-ethyl-4,5,6,7-tetrahydro-1H-imidazo[4,5-c]pyridine-6-carboxylic Acid:

A solution of (4S,6S)-4-Ethyl-4,5,6,7-tetrahydro-3H-imidazo[4,5-c]pyridine-6-carboxylic acid (500 mg, 2.56 mmol) in an aq. buffer solution at pH 9 (5 mL) and dioxane (1 mL) was cooled to 0° C. Benzyl chloroformate (0.845 mL, 5.64 mmol) was added dropwise over 1 min. The pH was maintained between 7 and 9 by adding from time to time aq. 1M NaOH (total amount: about 5 mL). The mixture was stirred overnight while the temperature rose slowly to room temperature. The final pH was equal to 6.5 and an oily precipitate was lying in the bottom of the flask. This oily precipitate was decanted, dissolved in CHCl₃ and the solution dried over MgSO₄. After filtration, the filtrate was evaporated under reduced pressure. The residue was diluted in MeOH (20 mL) and aq. 1M NaOH was added (10 mL). This solution was stirred at room temperature overnight and the pH was brought to 7 with aq. 1M HCl. The solvent was removed under reduced pressure and the residue dried under high vacuum. The dried residue was purified by RP—HPLC(C₁₈-column, 95% H₂O→75% H₂O over 10 min→0% H₂O over 10 min). The acid was obtained as a foam (224 mg, 40%). ¹H—NMR (400 MHz, CD₃OD): δ=8.15 (s, br., 1H); 7.40-7.23 (m, 5H); 5.46 (s, br., 1H); 5.21 (s, br., 3H); 2.83 (dd, J₁=15.6 Hz, J₂=6.1 Hz, 1H); 1.79 (s, br., 2H); 1.05 (s, br., 3H). MS (pos. ionisation): m/z=330 (MH⁺).

### EXAMPLE 1

4-Methyl-4,5,6,7-tetrahydro-1H-imidazo[4,5-c]Pyridine Dihydrochloride:

According to GPI with histamine dihydrochloride (12.1 g, 0.109 mol), NaOH (10.9 g, 0.272 mol), water (100 mL), MeOH (450 mL) and acetaldehyde (15.5 mL, 0.276 mol). The solution was acidified to pH<1 with aq. conc. HCl and the solvent was removed under reduced pressure. The residue was thoroughly dried under high vacuum. The resulting oil was triturated with MeOH (1×150 mL, 2×50 mL) and filtered. The filtrate was evaporated under reduced pressure and the residue dried under high vacuum. This residue was then suspended in ⁱPrOH and this suspension refluxed for 1 h. After allowing it to cool to room temperature, the mixture was filtered and the precipitate dried under high vacuum. The product was obtained as a brown powder that was not purified further (21.2 g, 90%). ¹H—NMR (400 MHz, D₂O): δ=8.70 (s, br., 1H); 3.76 (m, br., 1H); 3.53 (m, br., 1H); 3.09 (s, br., 2H); 1.69 (s, br., 3H).

### EXAMPLE 2

4-Ethyl-4,5,6,7-tetrahydro-1H-imidazo[4,5-c]Pyridine Dihydrochloride:

According to GPI with histamine dihydrochloride (20.0 g, 0.109 mol), NaOH (19.6 g, 0.49 mol), water (100 mL), MeOH (450 mL) and propionaldehyde (20.0 mL, 0.276 mol). The reaction mixture was acidified to pH<1 with aq. conc. HCl (200 mL) and the solvent was removed under reduced pressure. The residue was dried thoroughly under high vacuum and the resulting oil triturated with MeOH (1×150 mL and 2×50 mL). The filtrate was evaporated under reduced pressure and the residue dried under high vacuum. The resulting oil was triturated with EtOH (1×50 mL and 2×15 mL) and filtered. The precipitate was dried under high vacuum. 4-Ethyl-4,5,6,7-tetrahydro-1H-imidazo[4,5-c]pyridine dihydrochloride was obtained as a colorless powder (5.38 g, 22%). ¹H—NMR (400 MHz, CD₃OD): δ=8.95 (s, 1H); 4.69-4.61 (m, 1H); 3.80-3.70 (m, 1H); 3.55-3.46 (m, 1H); 3.20-3.02 (m, 2H); 2.31-2.15 (m, 1H); 2.05-1.92 (m, 1H); 1.15 (t, J=7.8 Hz, 3H). ¹³C—NMR (100 MHz, D₂O): δ=134.70 (d); 124.98 (s); 123.72 (s); 53.26 (d); 40.59 (t); 24.41 (t); 18.11 (t); 8.88 (q). MS (EI): m/z=151 (M⁺, 1%); 150 (2%); 122 (100%); 107 (5%); 95 (13%); 80 (3%). HRMS: calc. for C₈H₁₃N₃: M⁺=151.1158; found: 151.1109.

### EXAMPLE 3

4-Propyl-4,5,6,7-tetrahydro-1H-imidazo[4,5-c]Pyridine Dihydrochloride:

According to GPI with histamine dihydrochloride (20.0 g, 0.109 mol), NaOH (19.6 g, 0.49 mol), water (100 mL), MeOH (450 mL) and butyraldehyde (24.9 mL, 0.278 mol). The reaction mixture was acidified to pH<1 with aq. conc. HCl (200 mL) and the solvent was removed under reduced pressure. The residue was dried thoroughly under high vacuum and the resulting oil triturated with MeOH (1×150 mL and 2×50 mL). The filtrate was evaporated under reduced pressure and the residue dried under high vacuum. The resulting oil was triturated with EtOH (1×50 mL and 2×15 mL) and filtered. The precipitate was dried under high vacuum. 4-Propyl-4,5,6,7-tetrahydro-1H-imidazo[4,5-c]pyridine dihydrochloride was obtained as a colorless powder (20.9 g, 80%). ¹H—NMR (400 MHz, CD₃OD): δ=8.99 (s, 11H); 4.75 (dd, J₁=9.0 Hz, J₂=4.4 Hz, 1H); 3.82-3.73 (m, 1H); 3.59-3.50 (m, 1H); 3.24-3.05 (m, 2H); 2.21-2.10 (m, 1H); 2.04-1.90 (m, 1H); 1.70-1.52 (m, 2H); 1.07 (t, J=7.8 Hz, 3H). ¹³C—NMR (100 MHz, CD₃OD): δ=136.13 (d); 126.43 (s); 125.61 (s); 53.05 (d); 41.79 (t); 34.41 (t); 19.45 (t); 19.30 (t); 14.01 (q). MS (EI): m/z=165 (M⁺, 1%); 164 (2%); 135 (2%); 122 (100%); 95 (12%); 80 (2%); 68 (5%).

### EXAMPLE 4

4-Phenyl-4,5,6,7-tetrahydro-1H-imidazo[4,5-c]Pyridine Dihydrochloride:

According to GPI with histamine (10.0 g, 90 mmol), NaOH (9.0 g, 225 mol), water (90 mL), MeOH (365 mL) and benzaldehyde (23.1 mL, 0.23 mol). The reaction mixture was acidified to pH<1 with aq. conc. HCl (165 mL) and the solvent was removed under reduced pressure. The residue was dried thoroughly under high vacuum and the resulting oil triturated with hot ⁱPrOH (150 mL). The mixture was filtered and the precipitate washed with cold ⁱPrOH (2×30 mL). The filtrate was heated to reflux and hexane (210 mL) added slowly, whereas a precipitate formed. The mixture was cooled to room temperature, then to −18° C. The precipitate was filtered and dried. 4-Phenyl-4,5,6,7-tetrahydro-1H-imidazo[4,5-c]pyridine dihydrochloride was obtained as a yellow powder that contained 0.7 eq of ⁱPrOH (14.1 g, 12.2 g of product, 50%). ¹H—NMR (400 MHz, CD₃OD): δ=8.96 (s, 1H); 7.6-7.3 (m, 5H); 5.99 (s, 1H); 3.75-3.60 (m, 2H); 3.38-3.30 (m, 1H); 3.20 (dt, J₁=16.6 Hz, J₂=5.5 Hz, 1H). ¹³C—NMR (100 MHz, D₂O): δ=135.25 (s); 131.33 (d); 129.84 (d); 129.45 (d); 126.47 (s); 122.81 (s); 55.45 (d); 40.18 (t); 18.17 (t). MS (EI): m/z=199 (M⁺, 5%); 170 (100%); 122 (89%); 91 (3%). HRMS; Calc. for C₁₂H₁₃N₃: M⁺=199.1107; found: 199.1109.

### EXAMPLE 5

4-Benzyl-4,5,6,7-tetrahydro-1H-imidazo[4,5-c]Pyridine Dihydrochloride (3):

According to GPI with histamine dihydrochloride (20.0 g, 0.109 mol), NaOH (19.6 g, 0.49 mol), water (100 mL), MeOH (450 mL) and phenacetaldehyde (36 mL, 0.276 mol). The reaction mixture was acidified to pH<1 with aq. conc. HCl (50 mL) and the solvent was removed under reduced pressure. The residue was dried thoroughly under high vacuum and the resulting oil triturated with MeOH (1×150 mL and 2×50 mL). The filtrate was evaporated under reduced pressure and the residue dried under high vacuum. The resulting oil was triturated with ⁱPrOH and filtered. The precipitate was dried under high vacuum. 4-Benzyl-4,5,6,7-tetrahydro-1H-imidazo[4,5-c]pyridine dihydrochloride was obtained as a colorless powder that still contained 1 eq. ⁱPrOH (31.9 g, 25 g of product, 96%). ¹H—NMR (400 MHz, D₂O): δ=8.76 (s, 1H); 7.52-7.43 (m, 3H); 7.40-7.35 (m, 2H); 5.07 (dd, J₁=9.1 Hz, J₂=5.7 Hz, 1H); 3.76 (dt, J₁=13.1 Hz, J₂=5.2 Hz, 1H); 3.63 (dd, J₁=14.4 Hz, J₂=5.6 Hz, 1H); 3.53 (ddd, J₁=13.9 Hz, J₂=7.6 Hz, J₃=6.1 Hz, 1H); 3.24 (dd, J₁=14.2 Hz, J₂=9.3 Hz, 1H); 3.16 (dd, J₁=12.9 Hz, J₂=7.8 Hz, 1H).

### EXAMPLE 6

Methyl 4-methyl-1,4,6,7-tetrahydro-5H-imidazo[4,5-c]pyridine-5-carboxylate:

According to GPII, starting from 4-methyl-4,5,6,7-tetrahydro-1H-imidazo[4,5-c]pyridine dihydrochloride (500 mg, 2.38 mmol), K₂CO₃ (690 mg, 5.00 mmol), CHCl₃ (6 mL), H₂O (3 mL) and methyl chloroformate (0.39 mL, 5.0 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Methyl 4-methyl-1,4,6,7-tetrahydro-5H-imidazo[4,5-c]pyridine-5-carboxylate was obtained as foam (270 mg, 58%). R_{f}=0.30 (MeOH/CHCl₃ 1:9). ¹H—NMR (400 MHz, CD₃OD): δ=7.52 (s, 1H); 4.99 (s, br., 1H); 4.30 (s, br., 1H); 3.71 (s, 3H); 3.15 (td, br., J₁=11.7 Hz, J₂=3.7 Hz, 1H); 2.67 (m, 1H); 2.54 (dd, J₁=15.6 Hz, J₂=4.2 Hz, 1H); 1.88 (d, J₁=6.8 Hz, 3H). ¹³C—NMR (100 MHz, CD₃OD): δ=157.47 (s); 135.38 (d); 134.32 (s); 126.41 (s); 53.37 (q); 49.43 (d); 38.71 (t); 23.50 (t); 19.28 (q). MS (EI): m/z=195 (M⁺, 11%); 180 (100%); 136 (10%); 120 (16%); 107 (22%). HRMS: Calc. for C₉H₁₃N₃O₂: M⁺=195.1008; found: 195.1010.

### EXAMPLE 7

Benzyl 4-ethyl-1,4,6,7-tetrahydro-5H-imidazo[4,5-c]pyridine-5-carboxylate Trifluoroacetate:

According to GPII, starting from 4-ethyl-4,5,6,7-tetrahydro-1H-imidazo[4,5-c]pyridine dihydrochloride (1.00 g, 4.48 mmol), K₂CO₃ (1.24 g, 9.0 mmol), CHCl₃ (10 mL), H₂O (8 mL) and benzyl chloroformate (1.36 mL, 9.0 mmol). After basic treatment for 1 h the mixture was acidified to pH 8-8.5 with aq. 1M HCl. The reaction mixture was extracted with CHCl₃ (4×) and the combined org. phases were dried over Na₂SO₄. The solvent was removed under reduced pressure. Twice successively, the residue was treated with aq. 1M HCl and the solvent removed under reduced pressure. The residue was then suspended in aq. 1M HCl (15 mL) and washed with Et₂O (2×). The aq. phase was evaporated under reduced pressure and the residue purified by RP—HPLC (95% H₂O→0% H₂O over 20 min. Benzyl 4-ethyl-1,4,6,7-tetrahydro-5H-imidazo[4,5-c]pyridine-5-carboxylate trifluoroacetate was obtained as foam (590 mg, 33%). ¹H—NMR (400 MHz, CD₃OD): δ=8.75 (s, 1H); 7.42-7.27 (m, 5H); 5.19 (s, br., 3H); 4.46 (m, br., 1H); 3.25 (m, br., 1H); 2.78 (m, br., 1H); 2.69 (dd, J₁=15.4 Hz, J₂=3.4 Hz, 1H); 1.00 (s, br., 3H). ¹³C—NMR (100 MHz, CD₃OD): δ=134.09 (d); 129.46 (d); 129.19 (d); 68.90 (t); 52.60 (d); 38.25 (t); 28.04 (t); 10.86 (q). MS (EI): m/z=285 (M⁺, 0.4%); 256 (45%); 194 (34%); 150 (5%); 120 (9%); 107 (5%); 91 (100%). HRMS: Calc. for C₁₆H₁₉N₃O₂: M⁺=285.1477; found: 285.1471. Elemental analysis: Calc. for C₁₆H₁₉N₃O₂.C₂HF₃O₂.H₂O: C 51.8%, H 5.3%, N 10.6%; found: C 52.0%, H 4.9%, N 9.8%.

### EXAMPLE 8

Benzyl 4-phenyl-1,4,6,7-tetrahydro-5H-imidazo[4,5-c]pyridine-5-carboxylate Trifluoroacetate:

According to GPII, starting from 4-phenyl-4,5,6,7-tetrahydro-1H-imidazo[4,5-c]pyridine dihydrochloride (800 mg, 2.54 mmol), K₂CO₃ (737 mg, 5.33 mmol), CHCl₃ (6 mL), H₂O (5 mL) and benzyl chloroformate (0.80 mL, 5.33 mmol). After basic treatment for 1 h the mixture was acidified to pH 8 with aq. 1M HCl. The reaction mixture was extracted with CHCl₃ (4×) and the combined org. phases were dried over Na₂SO₄. The solvent was removed under reduced pressure and the residue purified by RP—HPLC (95% H₂O 0% H₂O over 20 min). Benzyl 4-phenyl-1,4,6,7-tetrahydro -5H-imidazo[4,5-c]pyridine-5-carboxylate trifluoroacetate was obtained as foam (291 mg, 25%) that contained 0.65 eq. H₂O according to the elemental analysis. t_{R}=11.26 min. ¹H—NMR (400 MHz, CD₃OD): δ=8.83 (s, 1H); 7.48-7.27 (m, 10H); 6.48 (s, br., 1H); 5.26 (d, br., J=10.8 Hz, 1H); 5.18 (d, J=12.0 Hz, 1H); 4.38 (d, br., J=12.2 Hz, 1H); 3.20 (ddd, J₁=15.9 Hz, J₂=11.2 Hz, J₃=4.6 Hz, 1H); 2.96-2.78 (m, 2H). ¹³C—NMR (100 MHz, CD₃OD): δ=139.04 (s); 137.49 (s); 134.91 (d); 129.89-128.98 (several peaks, d and s); 127.85 (s); 69.11 (t); 54.16 (d); 38.12 (t); 22.24 (t). MS (EI): m/z=333 (M⁺, 9%); 242 (89%); 212 (4%); 198 (61%); 169 (27%); 91 (100%). Elemental analysis: Calc. for C₂₀H₁₉N₃O₂.C₂HF₃O₂.⅔H₂O: C 57.5%, H 4.7%, N 9.1%; found: C 57.5%, H 4.4%, N 9.1%.

### EXAMPLE 9

Benzyl 4-benzyl-1,4,6,7-tetrahydro-5H-imidazo[4,5-c]pyridine-5-carboxylate:

According to GPII, starting from 4-benzyl-4,5,6,7-tetrahydro-1H-imidazo[4,5-c]pyridine dihydrochloride (500 mg, 1.74 mmol), K₂CO₃ (507 mg, 3.67 mmol), CHCl₃ (6 mL), H₂O (3 mL) and benzyl chloroformate (0.55 mL, 3.67 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Benzyl 4-benzyl-1,4,6,7-tetrahydro-5H-imidazo[4,5-c]pyridine-5-carboxylate was obtained as foam (225 mg, 42%). R_{f}=0.31 (MeOH/CHCl₃ 1:9). ¹H—NMR (400 MHz, CD₃OD): δ=7.57 (s, 1H); 7.23, 7.15 and 7.03 (m, 10H, rot.); 5.33, 5.25, 5.08 and 4.95 (m, 2H, rot.); 4.84 and 4.60 (d, J=12.2 Hz, 1H, rot.); 4.81 and 4.17 (dd, J₁=13.2 Hz and 13.7 Hz, J₂=5.4 Hz and 4.6 Hz, 1H, rot.); 3.24-2.90 (m, 3H, rot.); 2.83-2.33 (m, 2H, rot.). ¹³C—NMR (100 MHz, CD₃OD): δ=157.24 and 156.95 (s, rot.); 139.24 and 138.79 (s, rot.); 137.46 (s); 135.51 and 135.46 (d, rot.); 130.44 (d); 129.39, 129.24, 129.13, 129.01, 128.93, 128.83 and 128.72 (3×d, rot.); 127.36 (d); 68.31 and 68.17 (t, rot.); 55.24 and 54.53 (d, rot.); 40.96 and 40.20 (t, rot.); 39.73 and 38.97 (t, rot.); 23.39 and 22.88 (t, rot.). MS (EI): m/z=256 (M⁺—C₇H₇, 26%); 212 (22%); 91 (100%). HRMS: Calc. for C₂₁H₂₁N₃O₂: M⁺=347.1634; found: 347.1603.

### EXAMPLE 10

Benzyl 4-propyl-1,4,6,7-tetrahydro-5H-imidazo[4,5-c]pyridine-5-carboxylate Trifluoroacetate:

According to GPII, starting from 4-propyl-4,5,6,7-tetrahydro-1H-imidazo[4,5-c]pyridine dihydrochloride (600 mg, 2.52 mmol), K₂CO₃ (720 mg, 5.2 mmol), CHCl₃ (6 mL), H₂O (5 mL) and benzyl chloroformate (0.78 mL, 5.2 mmol). After basic treatment for 1 h the mixture was acidified to pH 8.5 with aq. 1M HCl. The reaction mixture was extracted with CHCl₃ (3×) and the combined org. phases were dried over Na₂SO₄. The solvent was removed under reduced pressure and the residue purified by RP—HPLC (85% H₂O→0% H₂O over 20 min). Benzyl 4-propyl-1,4,6,7-tetrahydro-5H-imidazo[4,5-c]pyridine-5-carboxylate trifluoroacetate was obtained as foam that was crystallised from Et₂O (276 mg, 27%). Mp =166-7° C. t_{R}=8.46 min. ¹H—NMR (400 MHz, CD₃OD): δ=8.74 (s); 7.42-7.27 (m, 5H); 5.31 (m, br., 1H); 5.18 (s, br., 2H); 4.44 (m, br., 1H); 3.25 (m, br., 1H); 2.78 (m, br., 1H); 2.68 (dd, J₁=15.6 Hz, J₂=3.4 Hz, 1H); 1.76 (m, 2H); 1.44 (m, br., 2H); 0.98 (m, br., 3H). ¹³C—NMR (100 MHz, CD₃OD); δ=156.92 (s); 134.08 (d); 130-128 (several peaks, s and d); 68.93 (t); 51.13 (d); 38.15 (t); 36.74 (t); 22.16 (t); 20.27 (t); 14.07 (q). MS (EI): m/z=300 (M+H⁺, 20%); 256 (51%); 212 (60%); 208 (28%); 192 (3%); 164 (16%); 120 (12%); 91 (100%). Elemental analysis: Calc. for C₁₇H₂₁N₃O₂.C₂HF₃O₂: C 55.2%, H 5.4%, N 10.2%; found C 55.1%, H 5.1%, N 10.0%.

### EXAMPLE 11

Methyl 4-ethyl-1,4,6,7-tetrahydro-5H-imidazo[4,5-c]pyridine-5-carboxylate Trifluoroacetate:

According to GPII, starting from 4-ethyl-4,5,6,7-tetrahydro-1H-imidazo[4,5-c]pyridine dihydrochloride (600 mg, 2.68 mmol), K₂CO₃ (778 mg, 5.63 mmol), CHCl₃ (6 mL), H₂O (4 mL) and methyl chloroformate (0.436 mL, 5.63 mmol). After basic treatment for 1 h the mixture was acidified to pH 8 with aq. 1 M HCl. The reaction mixture was extracted with CHCl₃ (4×) and the combined org. phases were dried over Na₂SO₄. The solvent was removed under reduced pressure and the residue purified by RP—HPLC (95% H₂O over 5 min then 40% H₂O over 10 min). Methyl 4-ethyl -1,4,6,7-tetrahydro-5H-imidazo[4,5-c]pyridine-5-carboxylate trifluoroacetate was obtained as foam (71 mg, 8%). ¹H-NMR (400 MHz, CD₃OD): δ=8.78 (s, 1H); 4.19 (s, br., 1H); 4.43 (s, br., 1H); 3.75 (s, 3H); 3.22 (m, br., 1H); 2.79 (m, 1H); 2.69 (m, 1H); 1.86 (m, 2H); 1.03 (t, J=7.6 Hz, 3H). ¹³C—NMR (100 MHz, d₆-DMSO, at 70° C.): δ=157.77 (s); 134.06 (d); 129.55 (s); 127.63 (s); 53.74 (q); 52.52 (d); 38.09 (t); 27.92 (t); 22.00 (t); 10.81 (q). MS (EI): m/z=209 (M⁺, 4%); 180 (100%); 150 (4%); 120 (54%); 107 (11%); 94 (15%); 93 (20%); 59 (76%). HRMS: Calc. for C₁₀H₁₅N₃O₂: M⁺=209.1164; found: 209.1171.

### EXAMPLE 12

Methyl 4-propyl-1,4,6,7-tetrahydro-5H-imidazo[4,5-c]pyridine-5-carboxylate Trifluoroacetate:

According to GPII, starting from 4-propyl-4,5,6,7-tetrahydro-1H-imidazo[4,5-c]pyridine dihydrochloride (600 mg, 2.52 mmol), K₂CO₃ (720 mg, 5.2 mmol), CHCl₃ (6 mL), H₂O (4 mL) and methyl chloroformate (0.40 mL, 5.2 mmol). After basic treatment for 1 h the mixture was acidified to pH 8-9 with aq. 1 M HCl. The reaction mixture was extracted with CHCl₃ (4×) and the combined org. phases were dried over Na₂SO₄. The solvent was removed under reduced pressure and the residue purified by RP—HPLC (95% H₂O→40% H₂O over 15 min). Methyl 4-propyl-1,4,6,7-tetrahydro-5H-imidazo[4,5-c]pyridine-5-carboxylate trifluoroacetate was obtained as foam (134 mg, 16%). ¹H-NMR (400 MHz, CD₃OD): δ=8.78 (s); 5.29 (s, br., 1H); 4.39 (s, br., 1H); 3.73 (s, 3H); 3.25 (s, br., 1H); 2.80 (m, 1H); 2.70 (dd, J₁=15.9 Hz, J₂=3.9 Hz, 1H); 1.79 (m, 1H); 1.46 (m, 1H); 1.00 (t, J=7.3 Hz, 3H). ¹³C—NMR (100 MHz, d₆-DMSO, at 70° C.); δ=155.23 (s); 132.46 (d); 127.74 (s); 125.32 (s); 52.35 (q); 49.05 (d); 36.45 (t); 34.90 (t); 20.49 (t); 18.23 (t); 13.12 (q). MS (EI): m/z=223 (M⁺, 2%); 194 (0.5%); 192 (1%); 180 (100%); 164 (1%); 148 (1%); 121 (6%); 120 (13%); 107 (4%). HRMS: Calc. for C₁₁H₁₇N₃O₂: M⁺=223.1321; found: 223.1323.

### EXAMPLE 13

Methyl 4-phenyl-1,4,6,7-tetrahydro-5H-imidazo[4,5-c]pyridine-5-carboxylate Trifluoroacetate:

According to GPII, starting from 4-phenyl-4,5,6,7-tetrahydro-1H-imidazo[4,5-c]pyridine dihydrochloride (800 mg, 2.54 mmol), K₂CO₃ (737 mg, 5.33 mmol), CHCl₃ (8 mL), H₂O (5 mL) and methyl chloroformate (0.412 mL, 5.33 mmol). After basic treatment for 1 h the mixture was acidified to pH 8 with aq. 1 M HCl. The reaction mixture was extracted with CHCl₃ (4×) and the combined org. phases were dried over Na₂SO₄. The solvent was removed under reduced pressure and the residue purified by RP—HPLC (95% H₂O→40% H₂O over 15 min). Methyl 4-phenyl-1,4,6,7-tetrahydro-5H-imidazo[4,5-c]pyridine-5-carboxylate trifluoroacetate was obtained as foam (506 mg, 54%) that contained 1 eq water according to the elemental analysis. ¹H—NMR (400 MHz, CD₃OD): δ=8.84 (s); 7.47-7.24 (m, 5H); 6.46 (s, br., 1H); 4.34 (d, br., J=11.2 Hz, 1H); 3.79 (s, 3H); 3.18 (ddd, J₁=15.9 Hz, J₂=11.2 Hz, J₃=4.6 Hz, 1H); 2.96-2.86 (m, 1H); 2.82 (dd, J₁=15.6 Hz, J₂=3.7 Hz, 1H). ¹³C—NMR (100 MHz, d₆-DMSO, at 70° C.): δ=154.83 (s); 138.18 (s); 133.75 (d); 128.33 (d); 127.99 (d); 127.26 (d); 127.15 (s); 125.44 (s); 52.56 (q); 52.26 (d); 36.58 (t); 20.64 (t). MS (EI): m/z=257 (M⁺, 100%); 242 (27%); 226 (7%); 198 (22%); 180 (67%); 121 (6%); 120 (11%). HRMS: Calc. for C₁₄H₁₅N₃O₂: M⁺=257.1164; found: 257.1164. Elemental analysis: Calc. for C₁₄H₁₅N₃O₂.C₂HF₃O₂.H₂O: C 49.4%, H 4.7%, N 10.8%; found: C 49.8%, H 4.8%, N 10.4%.

### EXAMPLE 14

4-Ethyl-5-(phenoxyacetyl)-4,5,6,7-tetrahydro-1H-imidazo[4,5-c]pyridine Trifluoroacetate:

According to GPII, starting from 4-ethyl-4,5,6,7-tetrahydro-1H-imidazo[4,5-c]pyridine dihydrochloride (600 mg, 2.68 mmol), K₂CO₃ (778 mg, 5.63 mmol), CHCl₃ (6 mL), H₂O (4 mL) and phenoxyacetyl chloride (0.78 mL, 5.6 mmol). After basic treatment for 1 h the mixture was acidified to pH 9 with aq. 1M HCl. The reaction mixture was extracted with CHCl₃ (3×) and the combined org. phases were dried over Na₂SO₄. The solvent was removed under reduced pressure and the residue purified by RP—HPLC (85% H₂₀→0% H₂O over 20 min). 4-Ethyl-5-(phenoxyacetyl)-4,5,6,7-tetrahydro-1H-imidazo[4,5-c]pyridine trifluoroacetate was obtained as foam (490 mg, 46%) that contained 1.5 eq. H₂O according to the elemental analysis. t_{R}=5.95 min. ¹H—NMR (400 MHz, CD₃OD): δ=8.72 (s, 1H); 7.25 (t, J=7.3 Hz, 2H); 7.08-6.82 (m, 3H); 5.61 (dd, J₁=9.3 Hz, J₂=4.6 Hz, 0.9H, 1^{st} rot.); 4.90-4.70 (m, 2 and 0.2H, 2^{nd} rot.); 4.25 (dd, J₁=14.4 Hz, J₂=5.2 Hz, 0.9H, 1^{st} rot.); 3.52 (ddd, J₁=14.9 Hz, J₂=12.0 Hz, J₃=4.4 Hz, 0.9H, 1^{st} rot.); 3.17 (m, 0.1H, 2^{nd} rot.); 2.93 (m, 1H); 2.76 (m, 1H); 1.10 and 0.97 (t, J=7.3 Hz, 3H, 2 rot.). ³C—NMR (100 MHz, CD₃OD): δ=170.08 (s); 159.18 (s); 134.29 (d); 130.48 (d); 129.34 (s); 127.20 (s); 122.60 (d); 115.62 (d); 68.03 (t); 50.38 (d); 39.47 (t); 27.75 (t); 22.86 (t); 10.80 (q). MS (EI): m/z=285 (M⁺, 15%); 256 (60%); 192 (43%); 178 (4%); 150 (13%); 135 (20%); 107 (45%); 77 (100%). HRMS: Calc. for C₉₆H₁₉N₃O₂: M⁺=285.1477; found: 285.1472. Elemental analysis: Calc. for C₁₆H₁₉N₃O₂.C₂HF₃O₂.3/2H₂O: C 50.7%, H 5.4%, N 9.8%; found: C 50.6%, H 5.6%, N 9.4%.

### EXAMPLE 15

4-Propyl-5-(phenoxyacetyl)-4,5,6,7-tetrahydro-1H-imidazo[4,5-c] pyridine Trifluoroacetate:

According to GPII, starting from 4-propyl-4,5,6,7-tetrahydro-1H-imidazo[4,5-c]pyridine dihydrochloride (600 mg, 2.52 mmol), K₂CO₃ (720 mg, 5.2 mmol), CHCl₃ (6 mL), H₂O (4 mL) and phenoxyacetyl chloride (0.72 mL, 5.2 mmol). After basic treatment for 1 h the mixture was acidified to pH 8-9 with aq. 1M HCl. The reaction mixture was extracted with CHCl₃ (4×) and the combined org. phases were dried over Na₂SO₄. The solvent was removed under reduced pressure and the residue purified by RP—HPLC (80% H₂O 0% H₂O over 20 min. 4-Propyl-5-(phenoxyacetyl)-4,5,6,7-tetrahydro-1H-imidazo[4,5-c]pyridine trifluoroacetate was obtained as foam (307 mg, 30%) that contained 1.5 eq. H₂O according to elemental analysis. t_{R}=6.36 min. ¹H—NMR (400 MHz, CD₃OD): δ=8.74 (s, 1H); 7.30-7.10 (m, 2H); 6.99-6.85 (m, 3H); 5.70 (t, J=7.1 Hz, 0.9H, 1^{st} rot.); 4.97-4.71 (m, 2H and 0.2H, 2^{nd} rot.); 4.27 (dd, J₁=14.6 Hz, J₂=5.4 Hz, 0.9H, 1^{st} rot.); 3.54 (ddd, J₁=15.3 Hz, J₂=11.7 Hz, J₃=4.2 Hz, 0.9H, 1^{st} rot.); 3.19 (m, 0.1H, 2^{nd} rot.); 2.94 (ddd, J₁=16.9 Hz, J₂=11.7 Hz, J₃=5.4 Hz, 0.9H, 1st rot.); 2.78 (dd, J₁=16.1 Hz, J₂=3.7 Hz, 0.9H, 1^{st} rot.); 2.70 (m, 0.2H, 2^{nd} rot.); 1.88 (m, 0.11H, 2^{nd} rot.); 1.80 (m, 0.9H, 1^{st} rot.); 1.52 (m, 0.1H, 2^{nd} rot.); 1.39 (m, 0.9H, 1^{st} rot.); 1.00 and 0.95 (t, J=7.5 Hz, 3H). ¹³C—NMR (100 MHz, CD₃OD); δ=170.04 (s); 134.29 (d); 130.49 (d); 129.57 (s); 127.16 (s); 122.63 (d); 115.65 (d); 68.08 (t); 48.86 (d); 39.39 (t); 36.71 (t); 22.89 (t); 20.19 (t); 14.18 (q). MS (EI): m/z=299 (M⁺, 21%); 256 (100%); 206 (64%); 192 (4%); 135 (23%); 120 (40%); 107 (39%); 93 (15%). HRMS: Calc. for C₁₇H₂₁N₃O₂: M⁺=299.1634; found: 299.1630. Elemental analysis: Calc. for C₁₇H₂₁N₃O₂.C₂HF₃O₂.3/2H₂O: C 51.8%, H 5.7%, N 9.5%; found: C 51.9%, H 5.6%, N 9.2%.

### EXAMPLE 16

4-Phenyl-5-(phenoxyacetyl)-4,5,6,7-tetrahydro-1H-imidazo[4,5-c]pyridine Trifluoroacetate:

According to GPII, starting from 4-phenyl-4,5,6,7-tetrahydro-1H-imidazo[4,5-c]pyridine dihydrochloride (800 mg, 2.54 mmol), K₂CO₃ (737 mg, 5.33 mmol), CHCl₃ (8 mL), H₂O (6 mL) and phenoxyacetyl chloride (0.74 mL, 5.33 mmol). After basic treatment for 1 h the mixture was acidified to pH 8-9 with aq. 1M HCl. The reaction mixture was extracted with CHCl₃ (4×) and the combined org. phases were dried over Na₂SO₄. The solvent was removed under reduced pressure and the residue purified by RP—HPLC (85% H₂O 0% H₂O over 20 min). 4-Phenyl-5-(phenoxyacetyl)-4,5,6,7-tetrahydro-1H-imidazo[4,5-c]pyridine trifluoroacetate was obtained as foam (400 mg, 35%). t_{R}=6.93 min. ¹H—NMR (400 MHz, CD₃OD): δ=8.85 (s, 1H); 7.46-7.23 (m, 7H); 7.00-6.86 (m, 3H); 4.96-4.76 (m, 3H); 4.20 (dd, J₁=14.4 Hz, J₂=4.9 Hz, 1H); 3.45 (m, 11H); 3.06 (m, 11H); 2.91 (d, J=15.6 Hz, 11H). ¹³C—NMR (100 MHz, d₆-DMSO): δ=166.72 (s); 157.72 (s); 138.00 (s); 134.10 (d); 129.27 (d); 128.62 (d); 128.32 (d); 127.77 (d); 127.42 (s); 124.84 (s); 120.89 (d); 114.52 (d); 65.93 (t); 49.70 (d); 37.73 (t); 21.59 (t). MS (EI): m/z=333 (M⁺, 77%); 256 (4%); 240 (100%); 226 (13%); 198 (56%); 169 (48%); 135 (3%); 122 (8%); 120 (12%); 94 (19%); 77 (73%). HRMS: Calc. for C₂₀H₁₉N₃O₂: M⁺=333.1477; found: 333.1475.

### EXAMPLE 17

Cyclopentyl 4-ethyl-1,4,6,7-tetrahydro-5H-imidazo[4,5-c]pyridine-5-carboxylate Trifluoroacetate:

According to GPII, starting from 4-ethyl-4,5,6,7-tetrahydro-1H-imidazo[4,5-c]pyridine dihydrochloride (400 mg, 1.78 mmol), K₂CO3 (517 mg, 3.74 mmol), CHCl₃ (4 mL), H₂O (2 mL) and cyclopentyl chloroformate (511 mg, 3.74 mmol). After basic treatment for 1 h the mixture was acidified to pH 9 with aq. 1M HCl. The reaction mixture was extracted with CHCl₃ (3×) and the combined org. phases were dried over Na₂SO₄. The solvent was removed under reduced pressure and the residue purified by RP—HPLC (95% H₂O→65% H₂O over 10 min→0% H₂O over 10 min). Cyclopentyl 4-ethyl-1,4,6,7-tetrahydro-5H-imidazo[4,5-c]pyridine-5-carboxylate trifluoroacetate was obtained as foam (174 mg, 26%). t_{R}=10.14 min. ¹H—NMR (400 MHz, CD₃OD): δ=8.75 (s); 5.17 (s, br., 1H); 5.12 (t, J=5.4 Hz, 1H); 3.21 (s, br., 1H); 2.76 (m, 1H); 2.68 (dd, J₁=14.9 Hz, J₂=3.4 Hz, 1H); 1.95-1.58 (m, 1H); 1.02 (t, J=7.3 Hz, 3H). ¹³C—NMR (100 MHz, CD₃OD): δ=157.00 (s); 134.08 (d); 129.71 (s); 127.70 (s); 80.44 (d); 52.48 (d); 37.72 (t); 33.78 (t); 27.86 (t); 24.61 (t); 22.00 (t); 10.95 (q). MS (EI): m/z=263 (M⁺, 4%); 234 (48%); 194 (12%); 178 (12%); 166 (100%); 122 (33%); 120 (14%); 107 (5%), 93 (5%). HRMS: Calc. for C₁₄H₂₁N₃O₂: M⁺=263.1634; found: 263.1595.

### EXAMPLE 18

Cyclopentyl 4-propyl-1,4,6,7-tetrahydro-5H-imidazo[4,5-c]pyridine-5-carboxylate Trifluoroacetate:

According to GPII, starting from 4-propyl-4,5,6,7-tetrahydro-1H-imidazo[4,5-c]pyridine dihydrochloride (400 mg, 1.68 mmol), K₂CO3 (487 mg, 3.53 mmol), CHCl₃ (4 mL), H₂O (2 mL) and cyclopentyl chloroformate (482 mg, 3.53 mmol). After basic treatment for 1 h the mixture was acidified to pH 8-9 with aq. 1M HCl. The reaction mixture was extracted with CHCl₃ (4×) and the combined org. phases were dried over Na₂SO₄. The solvent was removed under reduced pressure and the residue purified by RP—HPLC (95% H₂O→65% H₂O over 10 min→0% H₂O over 10 min). Cyclopentyl 4-propyl-1,4,6,7-tetrahydro-5H-imidazo[4,5-c]pyridine-5-carboxylate trifluoroacetate was obtained as foam (106 mg, 27%). t_{R}=10.97 min. ¹H—NMR (400 MHz, CD₃OD): δ=8.75 (s, 1H); 5.25 (m, br., 1H); 5.11 (s, br., 1H); 4.38 (m, br., 1H); 3.22 (m, br., 1H); 2.77 (m, br., 1H); 2.68 (dd, J₁=15.9 Hz, J₂=4.4 Hz, 1H); 1.95-1.58 (m, 10H); 1.46 (m, 12H); 1.00 (t, J=7.3 Hz, 3H). ¹³C—NMR (100 MHz, CD₃OD): δ=134.05 (d); 129.87 (s); 127.67 (s); 80.47 (d); 51.03 (d); 38.10 (t); 36.99 (t); 33.79 (t); 24.59 (t); 21.91 (t); 20.34 (t); 14.12 (q). MS (EI): m/z=277 (M⁺, 1%); 234 (32%); 208 (9%); 192 (10%); 166 (74%); 164 (7%); 150 (4%); 135 (5%); 122 (36%); 120 (49%); 95 (15%); 69 (100%). HRMS: Calc. for C₁₅H₂₃N₃O₂: M⁺=277.1790; found: 277.1780.

### EXAMPLE 19

Cyclopentyl 4-phenyl-1,4,6,7-tetrahydro-5H-imidazo[4,5-c]pyridine-5-carboxylate Trifluoroacetate:

According to GPII, starting from 4-phenyl-4,5,6,7-tetrahydro-1H-imidazo[4,5-c]pyridine dihydrochloride (500 mg, 1.59 mmol), K₂CO3 (461 mg, 3.34 mmol), CHCl₃ (5 mL), H₂O (2.5 mL) and cyclopentyl chloroformate (456 mg, 3.34 mmol). After basic treatment for 1 h the mixture was acidified to pH 8-9 with aq. 1 M HCl. The reaction mixture was extracted with CHCl₃ (4×) and the combined org. phases were dried over Na₂SO₄. The solvent was removed under reduced pressure and the residue purified by RP—HPLC (95% H₂O→65% H₂O over 10 min→0% H₂O over 10 min). Cyclopentyl 4-phenyl-1,4,6,7-tetrahydro-5H-imidazo[4,5-c]pyridine-5-carboxylate trifluoroacetate was obtained as foam (162 mg, 24%) that contained 1 eq. H₂O according to the elemental analysis. t_{R}=10.35 min. ¹H—NMR (400 MHz, CD₃OD): δ=8.83 (s, 1H); 7.43-7.35 (m, 3H); 7.32-7.27 (m, 2H); 6.44 (s, br., 1H); 5.16 (s, br., 1H); 4.34 (s, br., 1H); 3.18 (m, 1H); 2.91 (dd, J₁=16.1 Hz, J₂=5.9 Hz, 1H); 2.83 (dd, J₁=16.4 Hz, J₂=4.9 Hz, 1H); 1.97-1.56 (m, 8H). ¹³C—NMR (100 MHz, CD₃OD): δ=139.24 (s); 134.90 (d); 129.90 (d); 129.84 (d); 129.43 (s); 128.89 (d); 127.38 (s); 80.75 (d); 54.01 (d); 38.01 (t); 33.84 (t); 33.71 (t); 24.58 (t); 22.18 (t). MS (EI): m/z=311 (M⁺, 56%); 242 (100%); 226 (24%); 198 (91%); 169 (72%); 166 (39%); 122 (13%). HRMS: Calc. for C₁₈H₂₁N₃O₂: M⁺=311.1634; found: 311.1625. Elemental analysis: Calc. for C₁₈H₂₁N₃O₂.C₂HF₃O₂H₂O: C 54.2%, H 5.5%, N 9.5%; found: C 54.1%, H 5.4%, N 9.4%.

### EXAMPLE 20

4-Fluorophenyl 4-ethyl-1,4,6,7-tetrahydro-5H-imidazo[4,5-c]pyridine-5-carboxylate Trifluoroacetate:

According to GPII, starting from 4-ethyl-4,5,6,7-tetrahydro-1H-imidazo[4,5-c]pyridine dihydrochloride (400 mg, 1.78 mmol), K₂CO₃ (517 mg, 3.74 mmol), CHCl₃ (4 mL), H₂O (2 mL) and 4-fluorophenyl chloroformate (0.49 mL, 3.7 mmol). After basic treatment for 1 h the mixture was acidified to pH 9 with aq. 1 M HCl. The reaction mixture was extracted with CHCl₃ (3×) and the combined org. phases were dried over Na₂SO₄. The solvent was removed under reduced pressure, the residue taken in aq. 1 M HCl and washed with Et₂O(1×). The aq. phase was evaporated under reduced pressure and the residue was purified by RP—HPLC (90% H₂O→65% H₂O over 10 min→5% H₂O over 10 min). 4-Fluorophenyl 4-ethyl-1,4,6,7-tetrahydro-5H-imidazo[4,5-c]pyridine-5-carboxylate trifluoroacetate was obtained as foam (64 mg, 9%). t_{R}=9.21 min. ¹H-NMR (400 MHz, CD₃OD): δ=8.80 (s, 1H); 7.12 (m, 4H); 5.81 (m, br., 1H); 4.55 (m, br., 1H); 3.44 (m, br., 1H); 2.97 (m, br., 1H); 2.81 (m, br., 1H); 1.96 (m, br., 2H); 1.12 (m, br., 3H). ¹³C—NMR (100 MHz, CD₃OD): δ=161.44 (sd, J_{F}=182 Hz); 155.77 (s); 148.38 (s); 134.25 (d); 129.36 (s); 127.63 (s); 124.33 (d); 116.76 (sd, J_{F}=17 Hz); 53.01 (d); 38.31 (t); 27.62 (t); 22.39 (t); 10.84 (q). MS (EI): m/z=289 (M⁺, 6%); 260 (100%); 178 (77%); 122 (12%); 120 (12%); 112 (7%); 95 (15%). HRMS: Calc. for C₁₅H₁₆FN₃O₂: M⁺=289.1227; found: 289.1228.

### EXAMPLE 21

4-Fluorophenyl 4-propyl-1,4,6,7-tetrahydro-5H-imidazo[4,5-c]pyridine-5-carboxylate Trifluoroacetate:

According to GPII, starting from 4-propyl-4,5,6,7-tetrahydro-1H-imidazo[4,5-c]pyridine dihydrochloride (400 mg, 1.68 mmol), K₂CO₃ (487 mg, 3.53 mmol), CHCl₃ (4 mL), H₂O (2 mL) and 4-fluorophenyl chloroformate (0.47 mg, 3.6 mmol). After basic treatment for 1 h the mixture was acidified to pH 8-9 with aq. 1M HCl. The reaction mixture was extracted with CHCl₃ (4×) and the combined org. phases were dried over Na₂SO₄. The solvent was removed under reduced pressure and the residue purified by FC (MeOH/CHCl₃ 2:98→5:95→10:90→1:1). 4-Fluorophenyl 4-propyl-1,4,6,7-tetrahydro-5H-imidazo[4,5-c]pyridine-5-carboxylate trifluoroacetate was obtained as foam (92 mg, 18%). R_{f}=0.20 (MeOH/CHCl₃ 1:9). ¹H—NMR (400 MHz, CDCl₃): δ=7.80 (s, br., 1H); 7.03 (m, br., 4H); 7.23 and 4.93 (m, 1H, rot.); 4.45 and 4.36 (m, 1H, rot.); 3.43-3.09 (m, 1H); 2.88-2.76, 2.69-2.46 and 2.32-2.18 (m, 2H, rot.); 1.93-1.33 (m, 2H, rot.); 0.98-0.87 (m, 3H, rot.). ¹³C—NMR (100 MHz, CDCl₃): δ=159.93 (sd, J_{F}=182 Hz); 154.43 (s); 147.15 (s); 134.15 (d); 123.09 (d); 115.88 (dd, J_{F}=18 Hz); 53.16 and 52.58 (d, rot.); 38.96 and 38.74 (t, rot.); 37.01 and 36.64 (t, rot.); 23.36 and 22.27 (t, rot.); 19.67 and 19.34 (t, rot.); 14.34 and 14.18 (q, rot.). MS (EI): m/z=303 (M⁺, 2%); 260 (100%); 224 (5%); 208 (7%); 150 (19%); 122 (17%); 112 (10%). HRMS: Calc. for C₁₆H₁₈FN₃O₂: M⁺=303.1383; found: 303.1392.

### EXAMPLE 22

Methoxy-ethyl 4-ethyl-1,4,6,7-tetrahydro-5H-imidazo[4,5-c]pyridine-5-carboxylate:

According to GPII, starting from 4-ethyl-4,5,6,7-tetrahydro-1H-imidazo[4,5-c]pyridine dihydrochloride (400 mg, 1.78 mmol), K₂CO₃ (517 mg, 3.74 mmol), CHCl₃ (4 mL), H₂O (2 mL) and methoxyethyl chloroformate (518 mg, 3.74 mmol). After basic treatment for 1 h the mixture was acidified to pH 9 with aq. 1M HCl. The reaction mixture was extracted with CHCl₃ (3×) and the combined org. phases were dried over Na₂SO₄. The residue was purified by FC (MeOH/CHCl₃ 2:98→5:95→10:90). Methoxy-ethyl 4-ethyl-1,4,6,7-tetrahydro-5H-imidazo[4,5-c]pyridine-5-carboxylate was obtained as foam (98 mg, 22%) that contained 0.5 eq. H₂O according to the elemental analysis. R_{f}=0.05 (MeOH/CHCl₃ 1:9). ¹H—NMR (400 MHz, CD₃OD): δ=7.55 (s, 1H); 5.00 (s, br., 1H); 4.35 (m, br., 1H); 4.24 (s, br., 2H); 3.60 (s, br., 2H); 3.35 (s, br., 3H); 3.18 (s, br., 1H); 2.71 (ddd, J₁=16.4 Hz, J₂=12.0 Hz, J₃=5.6 Hz, 1H); 2.53 (dd, J₁=15.4 Hz, J₂=3.4 Hz, 1H); 1.89 (m, 1H); 1.71 (m, 1H); 1.00 (t, J=7.3 Hz, 3H). ¹³C—NMR (100 MHz, CD₃OD): δ=157.50 (s); 135.22 (d); 133.67 (s); 126.64 (s); 71.86 (t); 65.80 (t); 59.06 (q); 54.81 (d); 39.18 and 38.88 (t, rot.); 28.49 and 28.27 (t, rot.); 23.59 and 23.07 (t, rot.); 11.14 (q). MS (EI): m/z=253 (M⁺, 6%); 224 (100%); 194 (4%); 166 (7%); 150 (3%); 122 (38%). HRMS: Calc. for C₁₂H₁₉N₃O₃: M⁺=253.1426; found: 253.1421. Elemental analysis: Calc. for C₁₂H₁₉N₃O₃.½H₂O: C 54.95%, H 7.68%, N 16.07%; found: C 55.07%, H 7.81%, N 15.78%.

### EXAMPLE 23

Methoxyethyl 4-propyl-1,4,6,7-tetrahydro-5H-imidazo[4,5-c]pyridine-5-carboxylate: According to GPII, starting from 4-propyl-4,5,6,7-tetrahydro-1H-imidazo[4,5-c]pyridine dihydrochloride (400 mg, 1.68 mmol), K₂CO₃ (487 mg, 3.53 mmol), CHCl₃ (4 mL), H₂O (2 mL) and methoxyethyl chloroformate (490 mg, 3.53 mmol). After basic treatment for 1 h the mixture was acidified to pH 8-9 with aq. 1 M HCl. The reaction mixture was extracted with CHCl₃ (4×) and the combined org. phases were dried over Na₂SO₄. The solvent was removed under reduced pressure and the residue purified by FC (MeOH/CHCl₃ 2:98→5:95→10:90). Methoxyethyl 4-propyl-1,4,6,7-tetrahydro-5H-imidazo[4,5-c]pyridine-5-carboxylate was obtained as foam (87 mg, 20%) that contained 0.25 eq. H₂O according to the elemental analysis. R_{f}=0.25 (MeOH/CHCl₃ 1:9). ¹H—NMR (400 MHz, CD₃OD): δ=7.51 (s, 1H); 5.08 (s, br., 1H); 4.32 (m, br., 1H); 4.23 (m, br., 2H); 3.59 (s, br., 2H); 3.36 and 3.34 (s, 3H, rot.); 3.17 (m, br., 1H); 2.70 (ddd, J₁=16.1 Hz, J₂=12.0 Hz, J₃=5.6 Hz, 1H); 2.53 (dd, J₁=15.4 Hz, J₂=3.2 Hz, 1H); 1.79 (m, 1H); 1.70 (m, 1H); 1.45 (m, 2H); 0.97 (t, J=7.6 Hz, 3H). ¹³C—NMR (100 MHz, CD₃OD): δ=157.44 (s); 135.22 (d); 133.93 (s); 126.82 (s); 71.84 (t); 65.79 (t); 59.05 (q); 53.30 (d); 39.10 and 38.80 (t, rot.); 37.71 and 37.46 (t, rot.); 23.59 and 23.06 (t, rot.); 20.49 (t); 14.41 (q). MS (EI): m/z=267 (M⁺, 7%); 224 (100%); 208 (6%); 192 (7%); 180 (12%); 166 (11%); 122 (62%). HRMS: Calc. for C₁₃H₂₁N₃O₃: M⁺=267.1581; found: 267.1582. Elemental analysis: Calc. for C₁₃H₂₁N₃O₃.¼H₂O: C 57.44%, H 7.97%, N 15.52%; found: C 57.28%, H 8.11%, N 15.22%.

### EXAMPLE 24

Methoxyethyl 4-phenyl-1,4,6,7-tetrahydro-5H-imidazo[4,5-c]pyridine-5-carboxylate:

According to GPII, starting from 4-phenyl-4,5,6,7-tetrahydro-1H-imidazo[4,5-c]pyridine dihydrochloride (500 mg, 1.59 mmol), K₂CO₃ (461 mg, 3.34 mmol), CHCl₃ (6 mL), H₂O (4 mL) and methoxyethyl chloroformate (462 mg, 3.34 mmol). After basic treatment for 1 h the mixture was acidified to pH 8-9 with aq. 1 M HCl. The reaction mixture was extracted with CHCl₃ (4×) and the combined org. phases were dried over Na₂SO₄. The solvent was removed under reduced pressure and the residue purified by FC (MeOH/CHCl₃ 2:98→5:95→10:90). Methoxyethyl 4-phenyl-1,4,6,7-tetrahydro-5H-imidazo[4,5-c]pyridine-5-carboxylate was obtained as foam (267 mg, 56%). R_{f}=0.26 (MeOH/CHCl₃ 1:9). ¹H—NMR (400 MHz, CD₃OD): δ=7.62 (s, 1H); 7.30 (m, 5H); 6.30 (s, br., 1H); 4.29 (m, br., 2H); 3.65 (m, br., 2H); 3.38 (s, 3H); 3.09 (m, 1H); 2.82 (m, br., 1H); 2.65 (m, br., 1H). ¹³C-NMR (100 MHz, CD₃OD): δ=157.09 (s); 141.43 (s); 135.89 (d); 131.30 (s); 129.25 (d); 129.03 (d); 128.77 (d); 128.34 (s); 77.81 (t); 65.99 (t); 59.08 (q); 56.09 (d); 38.95 and 38.81 (t, rot.); 23.51 and 23.07 (t, rot.). MS (EI): m/z=301 (M⁺, 24%); 242 (26%); 198 (41%); 115 (10%); 88 (11%). HRMS: Calc. for C₁₆H₁₉N₃O₃: M⁺=301.1426; found: 301.1419.

### EXAMPLE 25

Benzyl 4-methyl-1,4,6,7-tetrahydro-5H-imidazo[4,5-c]pyridine-5-carboxylate:

According to GPII, starting from 4-methyl-4,5,6,7-tetrahydro-1H-imidazo[4,5-c]pyridine dihydrochloride (500 mg, 2.38 mmol), K₂CO₃ (690 mg, 5.00 mmol), CHCl₃ (6 mL), H₂O (3 mL) and benzyl chloroformate (0.75 mL, 5.0 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Benzyl 4-methyl-1,4,6,7-tetrahydro-5H-imidazo[4,5-c]pyridine-5-carboxylate was obtained as foam (193 mg, 30%). Rf=0.25 (MeOH/CHCl₃ 1:9). ¹H—NMR (400 MHz, CD₃OD): δ=7.51 (s, 11H); 7.38-7.52 (m, 5H); 5.13 (m, br., 3H); 4.33 (d, br., J=10 Hz, 1H); 3.16 (t, br., J=11.2 Hz, 1H); 2.66 (m, br., 1H); 2.54 (d, br., J=12.9 Hz, 1H); 1.38 (d, J=6.8 Hz, 3H). ¹³C—NMR (100 MHz, CDCl₃): δ=155.43 and 155.12 (s, rot.); 136.49 (s); 134.00 (d); 132.85 (s); 128.45 (d); 127.98 (d); 127.67 (d); 125.94 and 124.83 (s, rot.); 67.30 (t); 48.36 (d); 37.81 (t); 23.19 and 22.51 (t, rot.); 19.56 and 19.14 (q, rot.). MS (EI): m/z=271 (M⁺, 0.5%); 256 (6%); 180 (82%); 136 (16%); 108 (14%); 91 (100%). HRMS: Calc. for C₁₅H₁₇N₃O₂: M⁺=271.1321; found: 271.1308.

### EXAMPLE 26

Benzyl 4-benzyl-1,4,6,7-tetrahydro-5H-imidazo[4,5-c]pyridine-5-carboxylate:

According to GPII, starting from 4-benzyl-4,5,6,7-tetrahydro-1H-imidazo[4,5-c]pyridine dihydrochloride (500 mg, 1.74 mmol), K₂CO₃ (507 mg, 3.67 mmol), CHCl₃ (6 mL), H₂O (3 mL) and benzyl chloroformate (0.55 mL, 3.67 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Benzyl 4-benzyl-1,4,6,7-tetrahydro-5H-imidazo[4,5-c]pyridine-5-carboxylate was obtained as foam (225 mg, 42%). R_{f}=0.31 (MeOH/CHCl₃ 1:9). ¹H—NMR (400 MHz, CD₃OD): δ=7.57 (s, 1H); 7.23, 7.15 and 7.03 (m, 10H, rot.); 5.33, 5.25, 5.08 and 4.95 (m, 2H, rot.); 4.84 and 4.60 (d, J=12.2 Hz, 1H, rot.); 4.81 and 4.17 (dd, J₁=13.2 Hz and 13.7 Hz, J₂=5.4 Hz and 4.6 Hz, 1H, rot.); 3.24-2.90 (m, 3H, rot.); 2.83-2.33 (m, 2H, rot.). ¹³C-NMR (100 MHz, CD₃OD): δ=157.24 and 156.95 (s, rot.); 139.24 and 138.79 (s, rot.); 137.46 (s); 135.51 and 135.46 (d, rot.); 130.44 (d); 129.39, 129.24, 129.13, 129.01, 128.93, 128.83 and 128.72 (3×d, rot.); 127.36 (d); 68.31 and 68.17 (t, rot.); 55.24 and 54.53 (d, rot.); 40.96 and 40.20 (t, rot.); 39.73 and 38.97 (t, rot.); 23.39 and 22.88 (t, rot.). MS (EI): m/z=256 (M⁺—C₇H₇, 26%); 212 (22%); 91 (100%). HRMS: Calc. for C₂₁H₂₁N₃O₂: M⁺=347.1634; found: 347.1603.

### EXAMPLE 27

4-Methyl-5-(phenoxyacetyl)-4,5,6,7-tetrahydro-1H-imidazo[4,5-c]Pyridine:

According to GPII, starting from 4-methyl-4,5,6,7-tetrahydro-1H-imidazo[4,5-c]pyridine dihydrochloride (500 mg, 2.38 mmol), K₂CO₃ (690 mg, 5.00 mmol), CHCl₃ (6 mL), H₂O (3 mL) and phenoxyacetyl chloride (0.69 mL, 5.0 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). 4-Methyl-5-(phenoxyacetyl)-4,5,6,7-tetrahydro-1H-imidazo[4,5-c]pyridine was obtained as foam (28 mg, 5%). R_{f}=0.12 (MeOH/CHCl₃ 1:9). ¹H—NMR (400 MHz, CD₃OD): δ=7.55 and 7.53 (s, 1H, rot.); 7.26 (t, J=7.1 Hz, 2H); 6.99-6.90 (m, 3H); 5.45 and 5.05 (q, J=6.6 Hz, 1H, rot.); 4.95-4.77 (m, 3H); 4.73 and 4.14 (dd, J₁=13.4 Hz and 14.2 Hz, J₂=5.4 Hz and 5.1 Hz, 1H, rot.); 3.46 and 3.12 (ddd, J₁=12.0 Hz and 12.9 Hz, J₂=12.0 Hz and 12.9 Hz, J₃=4.2 Hz and 4.9 Hz, 1H, rot.); 2.88-2.54 (m, 2H, rot.); 1.54 and 1.41 (d, J=6.6 Hz and 6.8 Hz, 3H, rot.). ¹³C—NMR (100 MHz, CDCl₃): δ=166.41 and 166.91 (s); 157.83 (s); 134.29 (d); 129.74 (d); 121.86 (d); 114.74 and 114.63 (d, rot.); 68.18 and 67.47 (t, rot.); 50.15 and 46.78 (d, rot.); 39.81 and 35.94 (t, rot.); 24.03 and 22.04 (t, rot.); 20.52 and 18.91 (q, rot.). MS (EI): m/z=271 (M⁺, 20%); 194 (1%); 178 (100%); 164 (10%); 135 (15%); 107 (44%); 94 (15%).

### EXAMPLE 28

Allyl 4-methyl-1,4,6,7-tetrahydro-5H-imidazo[4,5-c]pyridine-5-carboxylate:

According to GPII, starting from 4-methyl-4,5,6,7-tetrahydro-1H-imidazo[4,5-c]pyridine dihydrochloride (500 mg, 2.38 mmol), K₂CO₃ (690 mg, 5.00 mmol), CHCl₃ (6 mL), H₂O (3 mL) and allyl chloroformate (0.53 mL, 5.0 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Allyl 4-methyl-1,4,6,7-tetrahydro-5H-imidazo[4,5-c]pyridine-5-carboxylate was obtained as foam (284 mg, 54%). R_{f}=0.30 (MeOH/CHCl₃ 1:9). ¹H—NMR (400 MHz, CD₃OD): δ=7.53 (s, 1H); 6.00-5.85 (m, 1H); 5.29 (ddd, J₁=17.2 Hz, J₂=3.2 Hz, J₃=1.7 Hz, 1H), 5.19 (ddd, J₁=10.5 Hz, J₂=2.8 Hz, J₃=1.3 Hz, 1H); 5.11 (d, br., J=6.4 Hz, 1H); 4.60 (dd, J₁=3.9 Hz, J₂=1.2 Hz, 2H); 4.33 (dd, J₁=13.4 Hz, J₂=4.9 Hz, 1H); 3.16 (m, br., 1H); 2.68 (m, 1H); 2.55 (ddd, J₁=15.4 Hz, J₂=4.2 Hz, J₃=1.0 Hz, 1H); 1.39 (d, J=6.8 Hz, 3H). ¹³C—NMR (100 MHz, CD₃OD): δ=156.58 (s); 135.40 (d); 134.31 (s); 134.09 (d); 126.31 (s); 117.73 (t); 67.25 (t); 49.39 (d); 38.71 (t); 23.57 and 23.24 (t, rot.); 19.54 and 19.16 (q, rot.). MS (EI): m/z=221 (M⁺, 2%); 206 (27%); 180 (100%); 136 (30%); 120 (30%); 107 (38%). HRMS: Calc. for C₁₁H₁₅N₃O₂: M⁺=221.1164; found: 221.1160.

### EXAMPLE 29

Allyl 4-ethyl-3,4,6,7-tetrahydro-5H-imidazo[4,5-c]pyridine-5-carboxylate:

According to GPII, starting from benzyl-4-ethyl-1,4,6,7-tetrahydro-5H-imidazo[4,5-c]pyridine-5-carboxylate trifluoroacetate (500 mg, 2.23 mmol), K₂CO₃ (646 mg, 4.68 mmol), CHCl₃ (6 mL), H₂O (3 mL) and allyl chloroformate (0.50 mL, 4.7 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→15:85 over 25 min). Allyl 4-ethyl-3,4,6,7-tetrahydro-5H-imidazo[4,5-c]pyridine-5-carboxylate was obtained as foam (2.2 mg, 0.4%). The low yield is probably due to a leak during the FC. R_{f}=0.15 (MeOH/CHCl₃ 1:9). ¹H—NMR (400 MHz, CD₃OD): δ=7.52 (s, 1H); 5.95 (m, 1H); 5.29 (dd, J₁=17.3 Hz, J₂=1.2 Hz, 1H); 5.20 (dd, J₁=10.5 Hz, J₂=1.2 Hz, 1H); 4.99 (m, br., 1H); 4.60 (d, J=5.1 Hz, 2H); 4.34 (dd, J₁=13.4 Hz, J₂=4.6 Hz, 1H); 3.19 (m, br., 1H); 2.70 (dddd, J₁=15.6 Hz, J₂=12.0 Hz, J₃=5.6 Hz, J₄=1.2 Hz, 1H); 2.53 (ddd, J₁=15.4 Hz, J₂=4.2 Hz, J₃=1.0 Hz, 1H); 1.88 (dddd, J₁=15.1 Hz, J₂=15.1 Hz, J₃=7.6 Hz, J₄=4.6 Hz, 1H); 1.71 (m, 1H); 0.99 (t, J=7.6 Hz, 3H). MS (EI): m/z=235 (M⁺, 2%); 206 (100%); 194 (16%); 178 (5%); 162 (38%); 150 (4%). HRMS: Calc. for C₁₂H₁₇N₃O₂: M⁺=235.1321; found: 235.1330.

### EXAMPLE 30

Allyl 4-propyl-1,4,6,7-tetrahydro-5H-imidazo[4,5-c]pyridine-5-carboxylate:

According to GPII, starting from 4-propyl-4,5,6,7-tetrahydro-1H-imidazo[4,5-c]pyridine dihydrochloride (500 mg, 2.10 mmol), K₂CO₃ (609 mg, 4.40 mmol), CHCl₃ (6 mL), H₂O (3 mL) and allyl chloroformate (0.47 mL, 4.4 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Allyl 4-propyl-1,4,6,7-tetrahydro-5H-imidazo[4,5-c]pyridine-5-carboxylate was obtained as foam (68 mg, 13%). R_{f}=0.20 (MeOH/CHCl₃ 1:9). ¹H—NMR (400 MHz, CD₃OD): δ=7.51 (s, 1H); 5.96 (m, 1H); 5.29 (d, br., J=17.3 Hz, 1H); 5.19 (dd, J₁=10.5 Hz, J₂=1.2 Hz, 1H); 5.09 (s, br., 1H); 4.60 (m, 2H); 4.32 (d, br., J=13.4 Hz, 1H); 3.19 (d, br., J=11.5 Hz, 1H); 2.70 (m, 1H); 2.55 (dd, J₁=15.4 Hz, J₂4.2 Hz, 1H); 1.79 (m, 1H); 1.70 (m, 1H); 1.45 (m, 2H); 0.96 (t, J=7.3 Hz, 3H). ¹³C—NMR (100 MHz, CD₃OD): δ=157.43 (s); 135.38 (d); 134.26 (s); 134.14 (d); 126.51 (s); 118.02 and 117.75 (t, rot.); 67.36 (t); 53.46 and 53.30 (d, rot.); 39.10 and 38.81 (t, rot.); 37.71 and 37.45 (t, rot.); 23.62 and 22.97 (t, rot.); 20.51 (t); 14.34 (q). MS (EI): m/z=249 (M⁺, 1%); 206 (44%); 192 (2%); 162 (13%); 135 (4%); 120 (34%); 107 (7%). HRMS: Calc. for C₁₃H₁₉N₃O₂: M⁺=249.1477; found: 249.1468.

### EXAMPLE 31

2,2,2-Trichloroethyl 4-ethyl-1,4,6,7-tetrahydro-5H-imidazo[4,5-c]pyridine-5-carboxylate:

According to GPII, starting from 4-ethyl-4,5,6,7-tetrahydro-1H-imidazo[4,5-c]pyridine dihydrochloride (500 mg, 2.23 mmol), K₂CO₃ (646 mg, 4.68 mmol), CHCl₃ (6 mL), H₂O (3 mL) and 2,2,2-trichloroethyl chloroformate (0.64 mL, 4.68 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). 2,2,2-Trichloroethyl 4-ethyl-1,4,6,7-tetrahydro-5H-imidazo[4,5-c]pyridine-5-carboxylate was obtained as foam (168 mg, 23%). R_{f}=0.16 (MeOH/CHCl₃ 1:9). ¹H—NMR (400 MHz, CD₃OD): δ=7.53 (s, 1H); 5.05 (m, 1H); 4.97-4.72 (m, 2H); 4.39 (m, 1H); 3.25 (m, 1H); 2.76 (m, 1H); 2.58 (dd, J₁=15.4 Hz, J₂=3.9 Hz, 1H); 1.93 (m, 1H); 1.75 (m, 1H); 1.03 (m, 3H, rot.). ¹³C—NMR (100 MHz, CD₃OD): δ=155.57 (s); 135.38 (d); 76.22 and 76.02 (t, rot.); 55.53 (d); 39.47 (t); 28.57 and 28.23 (t, rot.); 23.70 and 22.89 (t, rot.); 11.40 and 11.11 (q, rot.). MS (EI): m/z=325, 327, 329 (M⁺, 2%); 296, 298, 300 (100%); 262 (3%); 178 (19%); 166 (14%); 133 (17%); 122 (38%); 120 (38%); 107 (9%). HRMS: Calc. for 10 C₁₁H₁₄C₁₃N₃O₂: M+325.0152; found: 325.0148.

### EXAMPLE 32

Allyl 4-benzyl-1,4,6,7-tetrahydro-5H-imidazo[4,5-c]pyridine-5-carboxylate: According to GPII, starting from 4-benzyl-4,5,6,7-tetrahydro-1H-imidazo[4,5-c]pyridine dihydrochloride (500 mg, 1.74 mmol), K₂CO₃ (507 mg, 3.67 mmol), CHCl₃ 15 (6 mL), H₂O (3 mL) and allyl chloroformate (0.39 mL, 3.67 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). Allyl 4-benzyl-1,4,6,7-tetrahydro-5H-imidazo[4,5-c]pyridine-5-carboxylate was obtained as foam (63 mg, 12%). R_{f}=0.31 (MeOH/CHCl₃ 1:9). ¹H—NMR (400 MHz, CD₃OD): δ=7.59 (s, 1H); 7.29-6.94 (m, 5H); 5.86 and 5.69 (m, 1H, rot.); 5.32 and 5.23 (m, 1H, rot.); 5.23-5.12 (m, 1H); 5.04 (d, br., J=12.7 Hz, 1H); 4.49 and 4.29 (m, 2H, rot.); 4.18 and 4.03 (dd, J₁=13.4 Hz and 12.7 Hz, J₂=4.9 Hz and 4.4 Hz, 1H, rot.); 3.24-2.38 (m, 5H, rot.). ¹³C—NMR (100 MHz, CD₃OD): δ=157.06 and 156.80 (s, rot.); 139.33 and 138.83 (s, rot.); 135.52 (d); 134.08, 133.78 and 133.49 (2×s, rot.); 130.47 (d); 129.21 and 129.12 (d, rot.); 127.54 and 127.32 (d, rot.); 117.67 and 117.55 (t, rot.); 67.17 (t); 55.19 and 54.45 (d, rot.); 40.99 and 40.24 (t, rot.); 39.65 and 38.85 (t, rot.); 23.36 and 22.92 (t, rot.). MS (EI): m/z=297 (M⁺, 0.05%); 240 (3%); 22 (14%); 206 (100%); 162 (21%); 120 (21%); 91 (50%). HRMS: Calc. for C₁₇H₁₉N₃O₂: M⁺=297.1477; found: 297.1448.

### EXAMPLE 33

2,2,2-Trichloroethyl -4-propyl-1,4,6,7-tetrahydro-5H-imidazo[4,5-c]pyridine-5-carboxylate:

According to GPII, starting from 4-propyl-4,5,6,7-tetrahydro-1H-imidazo[4,5-c]pyridine dihydrochloride (500 mg, 2.10 mmol), K₂CO₃ (609 mg, 4.40 mmol), CHCl₃ (6 mL), H₂O (3 mL) and 2,2,2-trichloroethyl chloroformate (0.61 mL, 4.4 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→15:85 over 25 min). 2,2,2-Trichloroethyl 4-propyl-1,4,6,7-tetrahydro-5H-imidazo[4,5-c]pyridine-5-carboxylate was obtained as foam that still contained 0.2 eq. H₂O according to the elemental analysis (196 mg, 27%). R_{f}=0.25 (MeOH/CHCl₃ 1:9). ¹H—NMR (400 MHz, CD₃OD): δ=7.53 (s, 1H); 5.15 (m, br., 1H); 4.85 (m, 2H); 4.36 (m, 1H); 3.26 (m, 1H); 2.76 (m, 1H); 2.58 (d, J=15.4 Hz, 1H); 2.30 (d, J=15.1 Hz, 1H); 1.83 (m, 1H); 1.73 (m, 1H); 1.47 (m, 2H); 0.98 (t, J=7.3 Hz, 1H). ¹³C—NMR (100 MHz, CD₃OD): δ=155.53 (s); 135.38 (d); 76.23 and 76.02 (t, rot.); 54.15 and 53.90 (s, rot.); 51.78 and 51.61 (d, rot.); 39.42 and 39.04 (t, rot.); 37.84, 37.37, 37.11 and 36.64 (t, rot.); 23.70, 22.86, 22.27 and 21.56 (t, rot.); 20.74, 20.60, 20.49 and 20.34 (t, rot.); 14.51, 14.45, 14.22 and 14.27 (q, rot.). MS (EI): m/z=296, 298 and 300 (100%); 262 (4%); 208 (10%); 192 (16%); 166 (24%); 137 (17%); 122 (38%); 95 (25%). HRMS: Calc. for C₁₂H₁₆C₁₃N₃O₂: M⁺=339.0308; found: 339.0318. Elemental analysis: Calc. for C₁₂H₁₆C₁₃N₃O₂.⅕H₂O: C 41.87%, H 4.80%, N 12.21%; found: C 41.87%, H 4.84%, N 11.81%.

### EXAMPLE 34

2,2,2-Trichloroethyl -4-propyl-1,4,6,7-tetrahydro-5H-imidazo[4,5-c]pyridine -5-carboxylate: According to GPII, starting from 4-propyl-4,5,6,7-tetrahydro-1H-imidazo[4,5-c]pyridine dihydrochloride (500 mg, 2.10 mmol), K₂CO₃ (609 mg, 4.40 mmol), CHCl₃ (6 mL), H₂O (3 mL) and 2,2,2-trichloroethyl chloroformate (0.61 mL, 4.4 mmol). The reaction was carried out in a parallel fashion in a test-tube closed with a screwed tap and stirred in a Stem-stirrer. The basic treatment was carried out in MeOH (6 mL) and aq. 1 M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→15:85 over 25 min). 2,2,2-trichloroethyl 4-propyl-1,4,6,7-tetrahydro-5H-imidazo[4,5-c]pyridine-5-carboxylate was obtained as foam that still contained 0.2 eq. H₂O according to the elemental analysis (196 mg, 27%). R_{f}=0.25 (MeOH/CHCl₃ 1:9). ¹H—NMR (400 MHz, CD₃OD): δ=7.53 (s, 1H); 5.15 (m, br., 1H); 4.85 (m, 2H); 4.36 (m, 1H); 3.26 (m, 1H); 2.76 (m, 1H); 2.58 (d, J=15.4 Hz, 1H); 2.30 (d, J=15.1 Hz, 1H); 1.83 (m, 1H); 1.73 (m, 1H); 1.47 (m, 2H); 0.98 (t, J=7.3 Hz, 1H). ¹³C—NMR (100 MHz, CD₃OD): δ=155.53 (s); 135.38 (d); 76.23 and 76.02 (t, rot.); 54.15 and 53.90 (s, rot.); 51.78 and 51.61 (d, rot.); 39.42 and 39.04 (t, rot.); 37.84, 37.37, 37.11 and 36.64 (t, rot.); 23.70, 22.86, 22.27 and 21.56 (t, rot.); 20.74, 20.60, 20.49 and 20.34 (t, rot.); 14.51, 14.45, 14.22 and 14.27 (q, rot.). MS (EI): m/z=296, 298 and 300 (100%); 262 (4%); 208 (10%); 192 (16%); 166 (24%); 137 (17%); 122 (38%); 95 (25%). HRMS: Calc. for C₁₂H₁₆C₁₃N₃O₂: M⁺=339.0308; found: 339.0318. Elemental analysis: Calc. for C₁₂H₁₆C₁₃N₃O₂.⅕H₂O: C 41.87%, H 4.80%, N 12.21%; found: C 41.87%, H 4.84%, N 11.81%.

### EXAMPLE 35

2,2,2-Trichloroethyl 4-benzyl-1,4,6,7-tetrahydro-5H-imidazo[4,5-c]pyridine -5-carboxylate:

According to GPII, starting from 4-benzyl-4,5,6,7-tetrahydro-1H-imidazo[4,5-c]pyridine dihydrochloride (500 mg, 1.74 mmol), K₂CO₃ (507 mg, 3.67 mmol), CHCl₃ (6 mL), H₂O (3 mL) and 2,2,2-trichloroethyl chloroformate (0.51 mL, 3.7 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→15:85 over 25 min 2,2,2-trichloroethyl 4-benzyl-1,4,6,7-tetrahydro-5H-imidazo[4,5-c]pyridine-5-carboxylate was obtained as foam (211 mg, 31%). R_{f}=0.22 (MeOH/CHCl₃ 1:9). ¹H—NMR (400 MHz, CD₃OD): 6 =7.61 (s, 1H); 7.27-7.07 (m, 5H); 5.86 and 5.12 (m, 1H, rot.); 4.79-4.63 (m, 1.5H, rot.); 4.83-4.16 (m, 1.5H, rot.); 3.28-3.22 (m, 1H, rot.); 3.20-2.85 (m, 2H, rot.); 2.73-2.63 (m, 1H, rot.); 2.49 and 2.20 (dd, J=15.4 Hz and 15.6 Hz, J₂=3.2 Hz and 3.9 Hz, 1H, rot.). ¹³C—NMR (100 MHz, CD₃OD): δ=155.32, 155.13 and 154.98 (s, rot.); 138.94, 138.66, 138.25 and 138.07 (s, rot.); 135.69 and 135.60 (d, rot.); 130.62 and 130.51 (d, rot.); 129.34, 129.21 and 129.06 (d, rot.); 127.69, 127.58 127.45 and 127.39 (d, rot.); 75.97 and 75.88 (t, rot.); 55.41 and 55.13 (s, rot.); 52.84 and 52.70 (d, rot.); 40.89, 40.26 and 40.18 (t, rot.); 39.88, 39.70, 39.50 and 39.29 (t, rot.); 23.42, 22.74, 22.06 and 21.53 (t, rot.). MS (EI): m/z 387 (M⁺, 0.3%); 296, 298 and 300 (100%); 262 (4%); 226 (2%); 166 (22%); 122 (35%). HRMS: Calc. for C₁₆H₁₆C₁₃N₃O₂: M⁺=387.0308; found: 387.0318. Elemental analysis: Calc. for C₁₆H₁₆C₁₃N₃O₂: C 49.44%, H 4.15%, N 10.81%; found: C 49.53%, H 4.29%, N 10.36%.

### EXAMPLE 36

4-Ethyl-5-(4-nitrobenzyl)-1,4,6,7-tetrahydro-5H-imidazo[4,5-c]pyridine-5-carboxylate:

According to GPII, starting from 4-ethyl-4,5,6,7-tetrahydro-1H-imidazo [4,5-c]pyridine dihydrochloride (500 mg, 2.23 mmol), K₂CO₃ (646 mg, 4.68 mmol), CHCl₃ (6 mL), H₂O (3 mL) and old 4-nitrophenyl chloroformate (1.01 g, 4.68 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). 4-Ethyl-5-(4-nitrobenzyl)-1,4,6,7-tetrahydro-5H-imidazo[4,5-c]pyridine-5-carboxylate was obtained as foam (23 mg, 4%). R_{f}=0.11 (MeOH/CHCl₃ 1:9). ¹H—NMR (400 MHz, CD₃OD): δ=8.17 (d, J=8.8 Hz, 2H); 7.62 (d, J=8.8 Hz, 2H); 7.51 (s, 1H); 3.88 (d, J=14.4 Hz, 1H); 3.74 (d, J=14.4 Hz, 1H); 3.43 (t, J=6.1 Hz, 1H); 3.11 (m, 1H); 2.77 (m, 2H); 2.46 (dt, J₁=15.4 Hz, J₂=4.5 Hz, 1H); 1.78 (m, 2H); 0.89 (t, J=7.3 Hz, 3H). ¹³C—NMR (100 MHz, CD₃OD): δ=149.30 (s); 148.35 (s); 134.81 (d); 130.54 (d); 124.23 (d); 60.23 (d); 57.61 (t); 46.45 (t); 27.43 (t); 20.98 (t); 10.72 (q). MS (EI): m/z=286 (M⁺, 0.4%); 257 (100%); 241 (1%); 227 (1%); 211 (8%); 136 (4%); 122 (7%); 120 (11%). HRMS: Calc. for C₁₅H₂₀N₄O₂: M⁺=286.1430; found: 286.1436.

### EXAMPLE 37

5-(4-Nitrobenzyl)-4-propyl-1,4,6,7-tetrahydro-5H-imidazo[4,5-c]pyridine-5-carboxylate:

According to GPII, starting from 4-propyl-4,5,6,7-tetrahydro-1H-imidazo[4,5-c]pyridine dihydrochloride (500 mg, 2.10 mmol), K₂CO₃ (609 mg, 4.40 mmol), CHCl₃ (6 mL), H₂O (3 mL) and old 4-nitrophenyl chloroformate (949 mg, 4.40 mmol). The reaction was carried out in a parallel fashion in a test-tube closed with a screwed tap and stirred in a Stem-stirrer. The basic treatment was carried out in MeOH (6 mL) and aq. 1M NaOH (4 mL). After 1 h the mixture was acidified with aq. 1 M HCl (3 mL). The reaction mixture was evaporated in a speed-vac overnight. The residue was triturated with CHCl₃ and filtered several times. The combined org. phases were evaporated in a speed-vac. The residue was purified by parallel FC with a gradient pump (MeOH/CHCl₃ 0:100→0:100 for 5 min, then→1:3 over 25 min). 5-(4-Nitrobenzyl)-4-propyl-1,4,6,7-tetrahydro-5H-imidazo[4,5-c]pyridine-5-carboxylate was obtained as foam (88 mg, 14%). Rf=0.24 (MeOH/CHCl₃ 1:9). ¹H—NMR (400 MHz, CD₃OD): δ=8.14 (d, J=8.8 Hz, 2H); 7.58 (d, J=8.8 Hz, 2H); 7.50 (s, 1H); 3.83 (d, J=14.4 Hz, 1H); 3.74 (d, J=14.4 Hz, 1H); 3.49 (t, J=5.9 Hz, 1H); 3.12 (m, 1H); 2.85-2.70 (m, 2H); 2.44 (ddd, J₁=15.9 Hz, J₂=5.1 Hz, J₃=3.9 Hz, 1H); 1.69 (m, 2H); 1.39 (m, 2H); 0.82 (t, J=7.3 Hz, 3H). ¹³C—NMR (100 MHz, CDCl₃): δ=148.03 (s); 147.07 (s); 133.47 (d); 132.38 (s); 129.28 (d); 126.14 (s); 123.53 (d); 57.83 (d); 56.83 (t); 45.02 (t); 36.37 (t); 20.00 (t); 19.40 (t); 14.31 (q). MS (EI): m/z=299 (M—H⁺, 0.5%); 257 (100%); 241 (1%); 211 (9%); 136 (4%); 122 (3%); 120 (9%); 106 (3%); 90 (8%). Elemental analysis: Calc. for C₁₆H₂₂N₄O₂: C 63.56%, H 7.33%, N 17.06%; found: C 63.00%, H 7.32%, N 17.17%.

### EXAMPLE 38

Benzyl (4S,6S)-4-ethyl-6-[(isobutylamino)-carbonyl]-1,4,6,7-tetrahydro-5H-imidazo[4,5-c]pyridine-5-carboxylate trifluoroacetate: (4S,6S)-5-[(Benzyloxy)carbonyl]-4-ethyl-4,5,6,7-tetrahydro-1H-imidazo[4,5-c]pyridine-6-carboxylic acid (99 mg, 0.30 mmol) was dissolved in DMF (2 mL). EDC.HCl (63 mg, 0.33 mmol), HOBt (45 mg, 0.33 mmol), DMAP (cat. amount) and isobutylamine (70 μL, 0.70 mmol) were added. The solution was stirred for 24 h. After removing the solvent under reduced pressure, the residue was purified by RP—HPLC (95% H₂O→65% H₂O over 10 min→0% H₂O over 10 min). Benzyl (4S,6S)-4-ethyl-6-[(isobutylamino)-carbonyl]-1,4,6,7-tetrahydro-5H-imidazo[4,5-c]pyridine-5-carboxylate trifluoroacetate was obtained as foam (38.8 mg, 34%). ¹H—NMR (400 MHz, CDCl₃): δ=8.46 (s, 1H); 7.43-7.31 (m, 5H); 6.50 (s, br., 1H); 5.36 (s, br., 1H); 5.24 (s, br., 3H); 3.35 (d, J=16.4 Hz, 1H); 3.05 (dt, J₁=19.8 Hz, J₂=6.7 Hz, 1H); 2.90 (s, br., 1H); 2.82 (dd, J₁=16.4 Hz, J₂=6.8 Hz, 1H); 1.87-1.74 (m, br., 1H); 1.69 (s, br., 1H); 1.61 (m, 1H); 0.86 (s, br., 9H). ¹³C—NMR (100 MHz, CDCl₃): δ=170.82 (s); 157.60 (s); 135.30 (s); 133.16 (d); 128.70 (d); 126.47 (s); 124.79 (s); 69.03 (t); 51.88 (d); 50.73 (d); 47.32 (t); 28.16 (d and t); 20.33 (t); 20.21 (q); 10.97 (q). MS (EI): m/z=384 (M⁺, 11%); 355 (20%); 311 (15%); 284 (6%); 249 (7%); 194 (3%); 150 (8%); 122 (9%); 91 (100%). HRMS: Calc. for C₂₁H₂₈N₄O₃: M⁺=384.2161; found: 384.2172.

### EXAMPLE 39

Benzyl (4S,6S)-6-(aminocarbonyl)-4-ethyl-1,4,6,7-tetrahydro-5H-imidazo[4,5-c]pyridine-5-carboxylate trifluoroacetate: To a solution of (4S,6S)-5-[(Benzyloxy)carbonyl]-4-ethyl-4,5,6,7-tetrahydro-1H-imidazo[4,5-c]pyridine-6-carboxylic acid (80 mg, 0.24 mmol) in DMF (2 mL) were added NH₃/dioxane (0.5M, 1.5 mL, 0.75 mmol), EDC.HCl (48 mg, 0.25 mmol), HOBt (34 mg, 0.25 mmol) and DMAP (cat. amount). The solution was stirred overnight and the solvent removed under reduced pressure. The residue was dried under high vacuum and purified by RP—HPLC (95% H₂O→65% H₂O over 10 min→0% H₂O over 10 min). Benzyl (4S,6S)-6-(aminocarbonyl) -4-ethyl-1,4,6,7-tetrahydro-5H-imidazo[4,5-c]pyridine-5-carboxylate trifluoroacetate was obtained as foam (32.6 mg, 40%). ¹H—NMR (400 MHz, CD₃OD): 6 8.76 (s, 1H); 7.47-7.21 (m, 5H); 5.48 (s, br., 1H); 5.33-5.10 (m, br., 3H); 3.24 (dd, J₁=16.1 Hz, J₂=0.7 Hz, 1H); 2.83 (ddd, J₁=16.4 Hz, J₂=6.8 Hz, J₃=1.7 Hz); 1.90-1.73 (m, 2H); 1.06 (s, br., 3H). MS (EI): m/z=328 (M⁺, 4%); 284 (2%); 210 (6%); 193 (14%); 176 (3%); 107 (6%); 91 (100%). HRMS: Calc. for C₁₇H₂₀N₄O₃: M⁺=328.1535; found: 328.1523.

SSAO Activity Assays

All assays were performed at room temperature with SSAO purified from human umbilical cord arteries. The enzyme activity was measured with two different methods, based on the detection of either hydrogen peroxide or the aldehyde that is formed from SSAO catalysis of its main substrates, primary amines.

Hydrogen Peroxide Detection

This method is based on the horseradish peroxidase catalyzed hydrogen peroxide oxidation of 10-acetyl-3,7-dihydroxyphenoxazine (Molecular Probes A-65 50), that yields a highly fluorescent product, resorufin. Briefly, 10 mM stock solution of substance in DMSO is serially diluted in 0.05 M sodium-potassium phosphate buffer. These dilutions are mixed with benzylamine (SSAO substrate) and a reagent solution consisting of SSAO enzyme, horse radish peroxidase (HRP) and 10-acetyl-3,7-dihydroxyphenoxazine. The final concentrations in the assay volume are 104 μM benzylamine, 219 μM 10-acetyl-3,7-dihydroxyphenoxazine, 1.1 U/ml HRP and a dilution of the SSAO preparation of 1/600. After two hours of incubation in flat-bottomed polystyrene microtiter plates, the fluorescence is measured at 560 ex/590 em. The inhibition is measured as % decrease of the signal compared to a control containing dilution of DMSO only (no substance).

Aldehyde Detection

SSAO activity is measured as increase of aldehyde formed from SSAO degradation of primary amines. Briefly, in conical glass centrifuge tubes, ¹⁴C-labeled benzylamine is mixed with substance dilutions (from 10 mM stock solution in DMSO) in 0.05 M sodium-potassium phosphate buffer (pH 7.8). Enzyme, also diluted in phosphate buffer, is added and incubation is performed at room temperature for 60 minutes. The reaction is stopped with 1 M HCl. The formed (1⁴C-labeled) aldehyde is separated from the likewise 1⁴C-labeled benzylamine through extraction with toluene:ethyl acetate and then transferred to liquid scintillation vials for measurement of radioactivity in a beta counter. The final concentrations in the assay volume are 150 μM benzylamine (0.037 MBq/μmol), and a dilution of the SSAO preparation of 1/150.The inhibition is measured as % decrease of the signal compared to control containing dilution of DMSO only (no substance).

Biological Activity

The compounds shown in Tables 2 and 3 were tested for biological activity, determined as percent inhibition of SSAO at 12 μM concentration of the test compounds. The compounds were shown to inhibit SSAO to from 10 to 97%.

TABLE 2

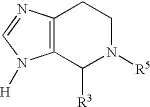

| Compound | R³ | R⁵ |
|---|---|---|
| 4-Methyl-4,5,6,7-tetrahydro-1H-imidazo[4,5-c]pyridine dihydrochloride | Me | H |
| 4-Ethyl-4,5,6,7-tetrahydro-1H-imidazo[4,5-c]pyridine dihydrochloride | Et | H |
| 4-Propyl-4,5,6,7-tetrahydro-1H-imidazo[4,5-c]pyridine dihydrochloride | Pr | H |
| 4-Phenyl-4,5,6,7-tetrahydro-1H-imidazo[4,5-c]pyridine dihydrochloride | Ph | H |
| 4-Benzyl-4,5,6,7-tetrahydro-1H-imidazo[4,5-c]pyridine dihydrochloride | Bn | H |
| Methyl 4-methyl-1,4,6,7-tetrahydro-5H-imidazo[4,5-c]pyridine-5-carboxylate | Me | Me—OCO— |
| Methyl 4-ethyl-1,4,6,7-tetrahydro-5H-imidazo[4,5-c]pyridine-5-carboxylate trifluoroacetate | Et | Me—OCO— |
| Methyl 4-propyl-1,4,6,7-tetrahydro-5H-imidazo[4,5-c]pyridine-5-carboxylate trifluoroacetate | Pr | Me—OCO— |
| Methyl 4-phenyl-1,4,6,7-tetrahydro-5H-imidazo[4,5-c]pyridine-5-carboxylate trifluoroacetate | Ph | Me—OCO— |
| Benzyl 4-methyl-1,4,6,7-tetrahydro-5H-imidazo[4,5-c]pyridine-5-carboxylate | Me | Bn—OCO— |
| Benzyl 4-ethyl-1,4,6,7-tetrahydro-5H-imidazo[4,5-c]pyridine-5-carboxylate trifluoroacetate | Et | Bn—OCO— |
| Benzyl 4-propyl-1,4,6,7-tetrahydro-5H-imidazo[4,5-c]pyridine-5-carboxylate trifluoroacetate | Pr | Bn—OCO— |
| Benzyl 4-phenyl-1,4,6,7-tetrahydro-5H-imidazo[4,5-c]pyridine-5-carboxylate trifluoroacetate | Ph | Bn—OCO— |
| Benzyl 4-benzyl-1,4,6,7-tetrahydro-5H-imidazo[4,5-c]pyridine-5-carboxylate | Bn | Bn—OCO— |
| 4-Methyl-5-(phenoxyacetyl)-4,5,6,7-tetrahydro-1H-imidazo[4,5-c]pyridine | Me | PhOCH₂CO— |
| 4-Ethyl-5-(phenoxyacetyl)-4,5,6,7-tetrahydro-1H-imidazo[4,5-c]pyridine trifluoroacetate | Et | PhOCH₂CO— |
| 4-Propyl-5-(phenoxyacetyl)-4,5,6,7-tetrahydro-1H-imidazo[4,5-c]pyridine trifluoroacetate | Pr | PhOCH₂CO— |
| 4-Phenyl-5-(phenoxyacetyl)-4,5,6,7-tetrahydro-1H-imidazo[4,5-c]pyridine trifluoroacetate | Ph | PhOCH₂CO— |
| Cyclopentyl 4-ethyl-1,4,6,7-tetrahydro-5H-imidazo[4,5-c]pyridine-5-carboxylate trifluoroacetate | Et | Cyclopentyl-OCO— |
| Cyclopentyl 4-propyl-1,4,6,7-tetrahydro-5H-imidazo[4,5-c]pyridine-5-carboxylate trifluoroacetate | Pr | Cyclopentyl-OCO— |
| Cyclopentyl 4-phenyl-1,4,6,7-tetrahydro-5H-imidazo[4,5-c]pyridine-5-carboxylate trifluoroacetate | Ph | Cyclopentyl-OCO— |
| 4-Fluorophenyl 4-ethyl-1,4,6,7-tetrahydro-5H-imidazo[4,5-c]pyridine-5-carboxylate trifluoroacetate | Et | 4F-Ph—OCO— |
| 4-Fluorophenyl 4-propyl-1,4,6,7-tetrahydro-5H-imidazo[4,5-c]pyridine-5-carboxylate trifluoroacetate | Pr | 4F-Ph—OCO— |
| 4-Fluorophenyl 4-phenyl-1,4,6,7-tetrahydro-5H-imidazo[4,5-c]pyridine-5-carboxylate trifluoroacetate | Ph | 4F-Ph—OCO— |
| Methoxyethyl 4-ethyl-1,4,6,7-tetrahydro-5H-imidazo[4,5-c]pyridine-5-carboxylate | Et | MeO(CH₂)₂—OCO— |
| Methoxyethyl 4-propyl-1,4,6,7-tetrahydro-5H-imidazo[4,5-c]pyridine-5-carboxylate | Pr | MeO(CH₂)₂—OCO— |
| Methoxyethyl 4-phenyl-1,4,6,7-tetrahydro-5H-imidazo[4,5-c]pyridine-5-carboxylate | Ph | MeO(CH₂)₂—OCO— |
| Allyl 4-methyl-1,4,6,7-tetrahydro-5H-imidazo[4,5-c]pyridine-5-carboxylate | Me | CH₂═CHCH₂—OCO— |
| Allyl 4-ethy1-3,4,6,7-tetrahydro-5H-imidazo[4,5-c]pyridine-5-carboxylate | Et | CH₂═CHCH₂—OCO— |
| Allyl 4-propyl-1,4,6,7-tetrahydro-5H-imidazo[4,5-c]pyridine-5-carboxylate | Pr | CH₂═CHCH₂—OCO— |
| Allyl 4-benzyl-1,4,6,7-tetrahydro-5H-imidazo[4,5-c]pyridine-5-carboxylate | Bn | CH₂═CHCH₂—OCO— |
| 2,2,2-Trichloroethyl 4-ethyl-1,4,6,7-tetrahydro-5H-imidazo[4,5-c]pyridine-5-carboxylate | Et | Cl₃CCH₂—OCO— |
| 2,2,2-Trichloroethyl 4-propyl-1,4,6,7-tetrahydro-5H-imidazo[4,5-c]pyridine-5-carboxylate | Pr | Cl₃CCH₂—OCO— |
| 2,2,2-Trichloroethyl 4-benzyl-1,4,6,7-tetrahydro-5H-imidazo[4,5-c]pyridine-5-carboxylate | Bn | Cl₃CCH₂—OCO— |
| 4-Ethyl-5-(4-nitrobenzyl)-1,4,6,7-tetrahydro-5H-imidazo[4,5-c]pyridine-5-carboxylate | Et | 4-NO₂—Ph—CH₂—OCO— |
| 5-(4-Nitrobenzyl)-4-propyl-1,4,6,7-tetrahydro-5H-imidazo[4,5-c]pyridine-5-carboxylate | Pr | 4-NO₂—Ph—CH₂—OCO— |

TABLE 3

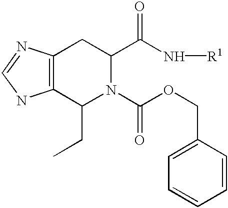

| Compound | R¹ |
|---|---|
| Benzyl(4S,6S)-4-ethyl-6-[(isobutyl- | i-Bu— |
| amino)-carbonyl]-1,4,6,7-tetra- | |
| hydro-5H-imidazo[4,5-c]pyridine-5-carboxylate trifluoroacetate | |
| Benzyl(4S,6S)-6-(aminocarbonyl)-4-ethyl-1,4,6,7-tetra- | H |
| hydro-5H-imidazo[4,5-c]pyridine-5-carboxylate-tri- | |
| fluoroacetate | |

What is claimed is:

1. A method of treatment or prophylaxis of SSAO-mediated complications in mammals including humans, comprising administering to a patient in need of such treatment a therapeutically effective amount of a compound of Formula (I): 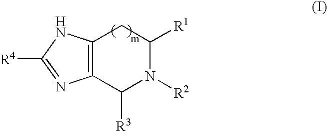
or a pharmaceutically acceptable salt thereof, wherein
R¹ is
(a) H, or
(b) CONH—R⁵,
R² is
(a) COOR⁵,
(b) COR⁵,
(c) CONH—R⁵,
(d) CSNH—R⁵, or
(e) H;
R³ is
(a) H,
(b) C₁₋₈ alkyl, or
(c) (CH₂)ₙAr;
R⁴is
(a) H,
(b) Ar, or
(c) C₁₋₈ alkyl; and
R⁵ is
(a) H,
(b) (CH₂)ₙAr,
(c) (CH₂)ₙOAr,
(d) C₁₋₈ alkyl containing 0-2 oxygen atoms and optionally substituted with 0-5 halogen atoms, or
(e) a polyether chain having the formula (CH₂)ₓO(CH₂)_{y}O(CH₂)_{z}CH₃;
n is an integer 0 to 4;
m is an integer 0 to 2;
x and y are integers 2 to 4;
z is an integer 0 to 3;
Ar is phenyl, 1-naphthyl or 2-naphthyl, unsubstituted optionally mono-or poly-substituted with electrodonating groups, halogen, C₁₋₆ alkyl, CF₃, hydroxyl, C₁₋₆ alkoxyl, OCF₃, CN, NO₂, phenyloxyl, benzyloxyl, optionally substituted phenyl, alkylsulfonyl, C₁₋₆ alkenyl, —NH₂, R⁷NH—, R⁷, R⁷N—, C₁₋₆ alkylcarboxyl, formyl, C₁₋₆ alkyl-CO—NH—, aminocarbonyl (R⁷, R⁷—N—CO—), SR⁷ wherein R⁷ is simultaneously or alternatively H or C₁₋₆ alkyl; cynnamoyl, unsubstituted or optionally substituted benzyl; 1,1-diphenylethyl, a monocyclic or bicyclic heterocyclic ring (furyl, pyrrolyl, triazolyl, diazolyl, oxazolyl, thiazolyl, oxadiazolyl, isothiazolyl, isoxazolyl, thiadiazolyl, pyridyl, pyrimidyl, pyrazinyl, thienyl, imidazolyl, pyrazolyl, indolyl, quinolinyl, isoquinolinyl, benzofuryl, benzothienyl, benzoxadiazolyl which are unsubstituted or optionally mono or di-substituted with halogen, C₁₋₆ alkyl); 2, or 3, or 4-pyridyl or a 5 to 7-membered unsaturated or partially or completely saturated heterocyclic ring each containing 1 to 4 heteroatoms selected from oxygen, nitrogen or sulfur where nitrogen containing heterocycles may contain H or C₁₋₆ alkyl or CF₃—CO— at the nitrogen atoms where such a substitution is allowed.

2. The method according to claim 1 wherein R¹ is H.

3. The method according to claim 1 wherein R²is COOR⁵.

4. The method according to claim 1 wherein R³ is C₁₋₃ alkyl or benzyl.

5. The method according to claim 1, wherein the compound of Formula (I) is selected from the group consisting of:
benzyl 4-methyl-1,4,6,7-tetrahydro-5H-imidazo[4,5-c]pyridine-5-carboxylate;
benzyl 4-ethyl-1,4,6,7-tetrahydro-5H-imidazo[4,5-c]pyridine-5-carboxylate trifluoroacetate;
benzyl 4-propyl-1,4,6,7-tetrahydro-5H-imidazo[4,5-c]pyridine-5-carboxylate trifluoroacetate;
2,2-Trichloroethyl 4-ethyl-1,4,6,7-tetrahydro-5H-imidazo[4,5-c]pyridine-5-carboxylate; and
benzyl (4S,6S)-6-(aminocarbonyl)-4-ethyl-1,4,6,7-tetrahydro-5H-imidazo[4,5-c]pyridine-5-carboxylate trifluoroacetate.

6. The method according to claim 1 for the treatment or prophylaxis of SSAO-mediated vascular complications.

7. The method according to claim 1 for the treatment or prophylaxis of diabetes.

8. A method of treatment or prophylaxis of SSAO-mediated complications in mammals including humans, comprising administering to a patient in need of such treatment a therapeutically effective amount of a compound of Formula (I): 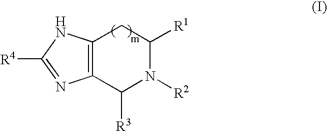
or a pharmaceutically acceptable salt thereof, wherein
R¹ is
(a) H, or
(b) CONH—R⁵;
R² is
(a) COOR⁵,
(b) COR⁵,
(c) CONH—R⁵, or
(d) CSNH—R⁵;
R³ is
(a) H,
(b) C₁₋₈ alkyl, or
(c) (CH₂),Ar;
R⁴ is
(a) H,
(b) Ar, or
(c) C₁₋₈ alkyl; and
R⁵ is
(a) H,
(b) (CH₂)ₙAr,
(c) (CH₂)ₙOAr,
(d) C₁₋₈ alkyl, or
(e) a polyether chain having the formula (CH₂)ₓO(CH₂)_{y}O(CH₂)_{z}CH₃;
n is an integer 0 to 4;
m is an integer 0 to 2;
x and y are integers 2 to 4;
z is an integer 0 to 3;
Ar is phenyl, 1-naphthyl or 2-naphthyl, unsubstituted optionally mono-or poly-substituted with halogen, C₁₋₆ alkyl, CF₃, hydroxyl, C₁₋₆ alkoxyl, OCF₃, CN, NO₂, phenyloxyl, benzyloxyl, optionally substituted phenyl, alkylsulfonyl, C₁₋₆ alkenyl, —NH₂, R⁷NH—, R⁷, R⁷N—, C₁₋₆ alkylcarboxyl, formyl, C₁₋₆ alkyl-CO—NH—, aminocarbonyl (R⁷, R⁷—N—CO—), SR⁷ wherein R⁷ is simultaneously or alternatively H or C₁₋₆ alkyl; cynnamoyl, unsubstituted or optionally substituted benzyl; 1,1-diphenylethyl, a monocyclic or bicyclic heterocyclic ring (furyl, pyrrolyl, triazolyl, diazolyl, oxazolyl, thiazolyl, oxadiazolyl, isothiazolyl, isoxazolyl, thiadiazolyl, pyridyl, pyrimidyl, pyrazinyl, thienyl, imidazolyl, pyrazolyl, indolyl, quinolinyl, isoquinolinyl, benzofuryl, benzothienyl, benzoxadiazolyl which are unsubstituted or optionally mono or di-substituted with halogen, C₁₋₆ alkyl); 2, or 3, or 4-pyridyl or a 5 to 7-membered unsaturated or partially or completely saturated heterocyclic ring each containing 1 to 4 heteroatoms selected from oxygen, nitrogen or sulfur where nitrogen containing heterocycles may contain H or C₁₋₆ alkyl or CF₃—CO— at the nitrogen atoms where such a substitution is allowed.

9. The method according to claim 8 for the treatment or prophylaxis of SSAO-mediated vascular complications.

10. The method according to claim 8 for the treatment or prophylaxis of diabetes.

11. A method of inhibiting SSAO activity in a mammal comprising administering to a patient in need of such treatment a therapeutically effective amount of a compound of Formula (I): 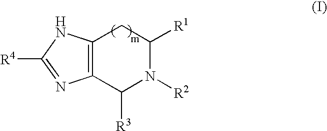
or a pharmaceutically acceptable salt thereof, wherein
R¹ is
(a) H, or
(b) CONH—R⁵;
R² is
(a) COOR⁵,
(b) COR⁵,
(c) CONH—R⁵, or
(d) CSNH—R⁵;
(e) H;
R³ is
(a) H,
(b) C₁₋₈ alkyl, or
(c) (CH₂)ₙAr;
R⁴ is
(a) H,
(b) Ar, or
(c) C₁₋₈ alkyl; and
R⁵ is
(a) H,
(b) (CH₂)ₙAr,
(c) (CH₂)ₙOAr,
(d) C₁₋₈ alkyl containing 0-2 oxygen atoms and optionally substituted with 0-5 halogen atoms, or
(e) a polyether chain having the formula (CH₂)ₓO(CH₂)_{y}O(CH₂)_{z}CH₃;
n is an integer 0 to 4;
m is an integer 0 to 2;
x and y are integers 2 to 4;
z is an integer 0 to 3;
Ar is phenyl, 1-naphthyl or 2-naphthyl, unsubstituted optionally mono-or poly-substituted with electrodonating groups, halogen, C₁₋₆ alkyl, CF₃, hydroxyl, C₁₋₆ alkoxyl, OCF₃, CN, NO₂, phenyloxyl, benzyloxyl, optionally substituted phenyl, alkylsulfonyl, C₁₋₆ alkenyl, —NH₂, R⁷NH—, R⁷, R⁷N—, C₁₋₆ alkylcarboxyl, formyl, C₁₋₆ alkyl-CO—NH—, aminocarbonyl (R⁷, R⁷—N—CO—), SR⁷ wherein R⁷ is simultaneously or alternatively H or C₁₋₆ alkyl; cynnamoyl, unsubstituted or optionally substituted benzyl; 1,1-diphenylethyl, a monocyclic or bicyclic heterocyclic ring (furyl, pyrrolyl, triazolyl, diazolyl, oxazolyl, thiazolyl, oxadiazolyl, isothiazolyl, isoxazolyl, thiadiazolyl, pyridyl, pyrimidyl, pyrazinyl, thienyl, imidazolyl, pyrazolyl, indolyl, quinolinyl, isoquinolinyl, benzofuryl, benzothienyl, benzoxadiazolyl which are unsubstituted or optionally mono or di-substituted with halogen, C₁₋₆ alkyl); 2, or 3, or 4-pyridyl or a 5 to 7-membered unsaturated or partially or completely saturated heterocyclic ring each containing 1 to 4 heteroatoms selected from oxygen, nitrogen or sulfur where nitrogen containing heterocycles may contain H or C₁₋₆ alkyl or CF₃—CO— at the nitrogen atoms where such a substitution is allowed.

12. The method according to claim 11, wherein the SSAO is overactive in the subject.

13. The method according to claim 1, wherein
R² is COOR⁵; and
R⁵ is:
1) H or a linear, branched or cyclic C₁₋₈ alkyl which can be saturated or not, containing 0-2 oxygen atoms and optionally substituted with 0-5 halogen atoms; or
2) (CH₂)ₙAr, where n=0-3 and Ar is a phenyl group or a phenyl group substituted with electrodonating groups, halogen atoms, or combination thereof.
